(19) United States
(12) Patent Application Publication
(10) Pub. No.: US 2007/0037983 A1
(43) Pub. Date: Feb. 15, 2007

(54) Phenanthroline derivative compound

(75) Inventors: Ryoji Nomura, Yamato (JP); Hideko Inoue, Atsugi (JP)

Correspondence Address: COOK, ALEX, McFARRON, MANZO,;CUMMINGS & MEHLER, LTD., SUITE 2850, 200 WEST ADAMS STREET, CHICAGO, IL, 60606, US

(73) Assignee: Semiconductor Energy Laboratory Co., Ltd.

(21) Appl. No.: 11/500,122

(22) Filed: Aug. 7, 2006

(30) Foreign Application Priority Data
Aug. 11, 2005 (JP) 2005-233382

(51) Int. Cl. C07D 471/02; H01L 51/54; H05B 33/12

(52) U.S. Cl. 546/88; 428/690; 428/917; 313/504; 313/506; 257/E51.049

(57) ABSTRACT

The present invention provides a novel phenanthroline derivative compound, and a manufacturing method thereof, and an electron transporting material, a light emitting element, a light emitting device, and an electronic device using the phenanthroline derivative compound. The phenanthroline derivative compound is characterized by being represented by General Formula 1. 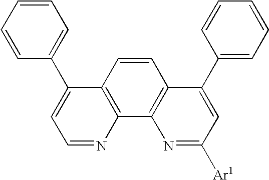
Note in General Formula 1 that Ar¹ represents an aryl group, and preferably a substituted or unsubstituted phenyl group, a substituted or unsubstituted naphthyl group, a substituted or unsubstituted anthryl group, or a substituted or unsubstituted phenanthryl group. As an example of a preferable compound, there is TMPBP, NaBP, PBP, or the like. A light emitting element, a light emitting device, and an electronic device preferably contain the compound in a light emitting layer, and in that case, the compound is preferably utilized as a host.

### BACKGROUND OF THE INVENTION

1. Field of the Invention

The present invention relates to a novel phenanthroline derivative compound and a manufacturing method thereof. Further, the present invention relates to an electron transporting material, a light emitting element, a light emitting device, and an electronic device using the phenanthroline derivative compound. In more detail, the present invention relates to a novel phenanthroline derivative compound preferable for a light emitting element, which is superior in an electron transporting property and does not cause crystallization, so that life of the element can be lengthened, and a manufacturing method thereof. Further, the present invention relates to an electron transporting material, a light emitting element, a light emitting device, and an electronic device using the phenanthroline derivative compound.

2. Description of the Related Art

In recent years, research and development of a light emitting element using a light-emitting organic compound called an electroluminescent element are actively carried out. This light emitting element is abbreviated as an organic EL element, and a basic structure thereof is such that a layer containing a light-emitting organic compound is interposed between a pair of electrodes. By applying a voltage to this element, electrons and holes are each injected to the layer containing a light-emitting organic compound from the pair of electrodes; accordingly, a current flows. Then, by recombining the carriers (electrons and holes), the light-emitting organic compound becomes in an excited state, and when the excited state returns to a ground state, light is emitted.

As a specific structure, there is typically a structure shown in FIG. 12 or 13, and FIG. 12 shows a two-layered structure in which a fluorescent thin film (light emitting layer) 3 and a hole transporting layer 4, which are stacked, are provided between a metal electrode 1 which is a cathode and a transparent electrode 2 which is an anode. In addition, FIG. 13 shows a three-layered structure in which an electron transporting layer 5, the light emitting layer 3, and the hole transporting layer 4, which are stacked, are provided between the metal electrode 1 and the transparent electrode 2. Here, the hole transporting layer 4 has a function of easily injecting holes from the anode and an electron blocking property, while the electron transporting layer 5 has a function of easily injecting electrons from the cathode.

In a light emitting element using this light-emitting organic compound, a glass substrate 6 is provided on the transparent electrode 2 on a side opposite to the hole transporting layer 4. Excitons are generated by the recombination of the electrons injected from the metal electrode 1 and the holes injected from the transparent electrode 2, the excitons emit light in a process of radiative deactivation, and this light is emitted through the transparent electrode 2 and the glass substrate 6 to the outside, so that light emission is performed. By such a mechanism, the above-described light emitting element is also called a current excitation type light emitting element. In addition, there is a single excited state or a triplet excited state as an excitation state formed by the organic compound, and light emitted from the single excited state is called fluorescence, and light emitted from the triplet excited state is called phosphorescence.

In this light emitting element, a light-emitting organic compound is naturally used for the light emitting layer, and in addition to this, organic compounds having appropriate properties to the hole transporting layer and the electron transporting layer respectively, are employed for the light emitting layer. For example, an organic compound having an electron transporting property is used for an electron transporting layer, and as the organic compound having such a property, various kinds of compounds such as phenanthroline and a derivative thereof have already proposed (Reference 1: Japanese Patent No. 3562652).

In such a situation, the present applicants has been dedicated to researching and developing a light emitting element which uses this light-emitting organic compound, and especially has been dedicated to researching and developing various organic compounds for a light emitting element.

### SUMMARY OF THE INVENTION

Accordingly, the present inventors have paid attention to characteristics of a phenanthroline derivative and have researched the characteristics. Then, it is found that a phenanthroline derivative is superior in an electron transporting property and can provide an element superior in an initial characterization. In addition to that, a phenanthroline skeleton itself has a wide band gap and has superior characteristics as a hole blocking layer.

As described above, a phenanthroline derivative has superior characteristics. However, the present inventors have researched and found that a known phenanthroline derivative has a disadvantage of having high crystallinity, which causes crystallization in an element, so that life of the light emitting element is shortened. The present inventors have researched and developed so as to solve this disadvantage and develop a novel phenanthroline derivative; and as a result, a phenanthroline derivative compound of the present invention is successively developed.

Therefore, it is an object of the present invention to provide a novel phenanthroline derivative without the above-described disadvantage and a manufacturing method thereof. In other words, it is an object of the present invention to provide a novel phenanthroline derivative, which has a low crystallinity so that crystallization is hardly caused and which can lengthen life of the light emitting element, and a manufacturing method thereof.

Further, it is another object of the present invention to provide a light emitting element superior in an electron transporting property as well as a known phenanthroline derivative, and superior in an initial characterization. In addition, it is another object of the present invention to provide a novel phenanthroline derivative having a phenanthroline skeleton, a wide band gap, and a superior property as an electron transporting layer or a hole blocking layer. It is another object of the present invention to provide an electron transporting material, a light emitting element, a light emitting device, and an electronic device utilizing the phenanthroline derivative having such a superior property.

The present invention provides a novel phenanthroline derivative compound which solves the above-described problems and a manufacturing method thereof, and an electron transporting material, a light emitting element, and an electronic device utilizing the above-described phenanthroline derivative compound. A novel phenanthroline derivative compound is represented by General Formula 1.

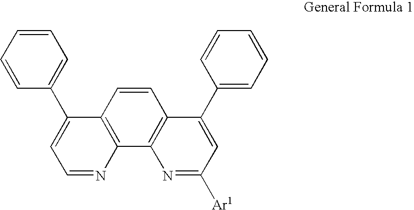

Note in General Formula 1 that Ar¹ represents an aryl group, and preferably a substituted or unsubstituted phenyl group, a substituted or unsubstituted naphthyl group, a substituted or unsubstituted anthryl group, or a substituted or unsubstituted phenanthryl group.

Further, the novel phenanthroline derivative compound is more preferably a material represented by any of following General Formulas 2 to 6.

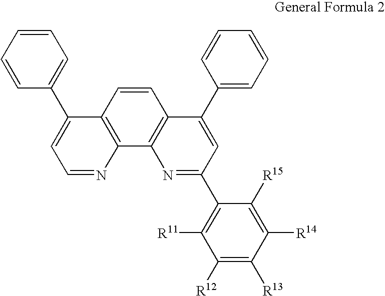

Note in General Formula 2 that R¹¹ to R¹⁵ each represent hydrogen, an alkyl group or an aryl group.

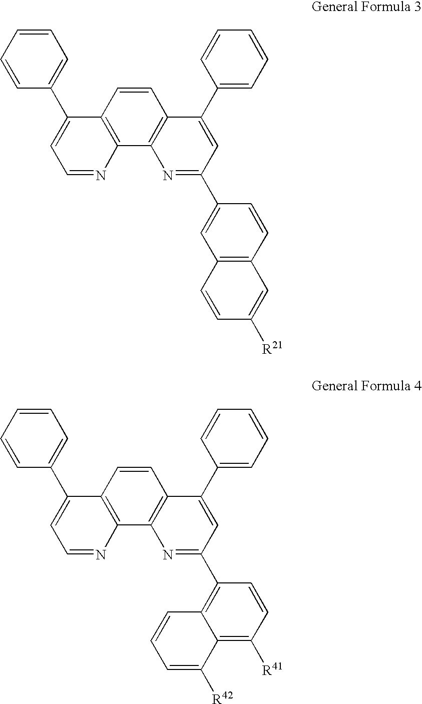

Note in General Formula 3 or 4 that R²¹ represents hydrogen, an alkyl group, or an aryl group, and R⁴¹ and R⁴² each represent hydrogen, an alkyl group or an aryl group.

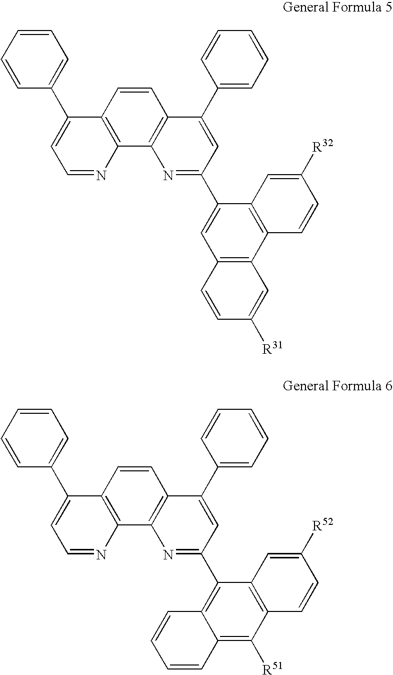

Note in General Formula 5 or 6 that R³¹, R³², R⁵¹, and R⁵² each represent hydrogen, an alkyl group or an aryl group.

In addition, a manufacturing method of the novel phenanthroline derivative compound of the present invention is as follows. That is, the manufacturing method is characterized by reacting aryl halide capable of forming a predetermined aryl group with Mg or BuLi, then performing a reaction with 4,7-diphenyl-1,10-phenanthroline, performing a reaction with water or alcohol to generate a 1:1 adduct, and eliminating hydrogen bonded with nitrogen at a first position and hydrogen bonded with carbon at a second position of the generated 1:1 adduct by using MnO₂.

An electron transporting material of the present invention contains the above-described compound. A light emitting element of the present invention includes a layer containing the above-described compound, which is preferably a light emitting layer, an electron transporting layer, or a blocking layer. In the case of forming the light emitting layer, the above-described compound is preferably utilized as a host of the light emitting layer, and in that case, in addition to a light emitting substance, another host material or a second substance for efficiently transferring excitation energy of the host to the light emitting substance may be contained. In addition, a light emitting device and an electronic device of the present invention include the above-described light emitting element of the present invention.

The phenanthroline derivative compound of the present invention has a superior electron transporting property, hardly causes crystallization, and as a result, has an excellent property of being able to lengthen life of an element, and also is a novel compound preferable for a light emitting element. The manufacturing method of the phenanthroline derivative compound of the present invention provides a method for manufacturing the compound having such excellent characteristics. Since the phenanthroline derivative compound of the present invention has excellent characteristics as described above, it can be utilized for an electron transporting material, a light emitting element, a light emitting device, and an electronic device.

In particular, a phenanthroline derivative compound of the present invention has a phenanthroline skeleton and a wide band gap, and can exhibit excellent characteristics as an electron transporting layer or a hole blocking layer when it is used as a light emitting element. Further, the phenanthroline derivative compound of the present invention is preferably used as a host in forming a light emitting layer, and in that case, in addition to a light emitting substance, another host material or a second substance for efficiently transferring excitation energy of the host to the light emitting substance may be contained. A host of a light emitting layer means a substance which does not emit light itself but efficiently injects carriers or provides excitation energy to a light emitting substance dispersed in the host.

### BRIEF DESCRIPTION OF DRAWINGS

In the accompanying drawings:

FIGS. 1A to 1C show stacked structures of light emitting elements of the present invention;

FIG. 2 shows a stacked structure having a different mode from those of FIGS. 1A to 1C in a light emitting element of the present invention;

FIG. 3 shows a stacked structure having a different mode from those of FIGS. 1A to 1C and 2 in a light emitting element of the present invention;

FIGS. 4A and 4B show a stacked structure of a light emitting device of the present invention;

FIG. 5 shows a stacked structure having a different mode from that in FIG. 4 in a light emitting device of the present invention;

FIGS. 6A to 6E are general pictures of electronic devices of the present invention;

FIG. 7 shows emission spectrums obtained by measuring films of NaBP and PBP manufactured in Embodiment 2;

FIG. 8 shows a voltage-luminance curve which is obtained by measuring a light emitting element using PBP manufactured in Embodiment 3;

FIG. 9 shows an emission spectrum which is obtained by measuring a light emitting element using PBP manufactured in Embodiment 3;

FIG. 10 shows a voltage-luminance curve obtained by measuring a light emitting element using BCP manufactured in Embodiment 3;

FIG. 11 shows an emission spectrum obtained by measuring a light emitting element using BCP manufactured in Embodiment 3;

FIG. 12 shows a stacked structure of an organic EL element in a conventional technique; and

FIG. 13 shows a stacked structure of an organic EL element different from that of FIG. 12 of a conventional technique.

### DETAILED DESCRIPTION OF THE INVENTION

Hereinafter, a phenanthroline derivative compound of the present invention and a manufacturing method thereof, and an electron transporting material, a light emitting element, a light emitting device, and an electronic device utilizing the phenanthroline derivative compound will be described in detail in embodiment modes; however the present invention is not limited to those embodiment modes and shall be specified by the description of the scope of claims.

### EMBODIMENT MODE 1

First, an embodiment mode of a compound of the present invention is hereinafter described in detail. A phenanthroline derivative compound of the present invention is a novel compound and represented by General Formula 1. Note in General Formula 1 that Ar¹ represents an aryl group, and preferably a substituted or unsubstituted phenyl group, a substituted or unsubstituted naphthyl group, a substituted or unsubstituted anthryl group, or a substituted or unsubstituted phenanthryl group.

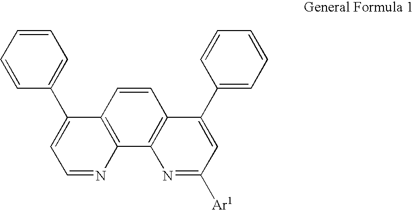

In addition, the novel phenanthroline derivative compound is preferably represented by any of following General Formulas 2 to 6.

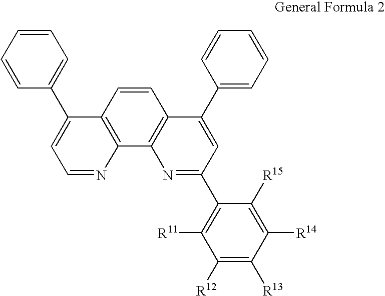

Note that in General Formula 2, R¹¹ to R¹⁵ each represent hydrogen, an alkyl group, or an aryl group. An alkyl group may be not only a straight-chain alkyl group but also a cyclic alkyl group, that is, a cycloalkyl group, and this point is not only for General Formula 2 and the same can applied to General Formulas 3 to 6.

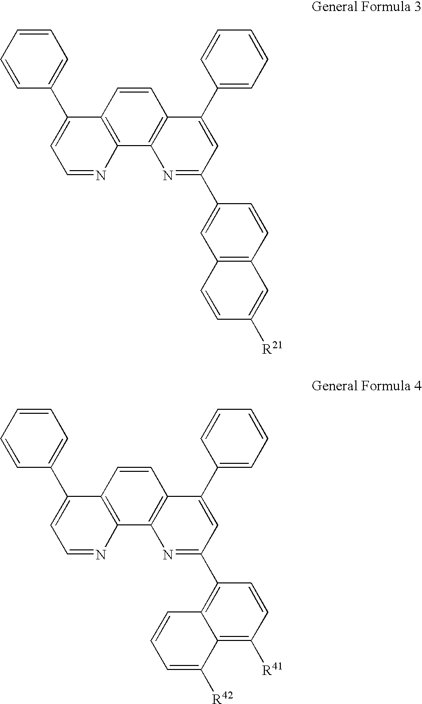

Note that in General Formula 3 or 4, R²¹ represents hydrogen, an alkyl group, or an aryl group, and R⁴¹ and R⁴² each represent hydrogen, an alkyl group, or an aryl group.

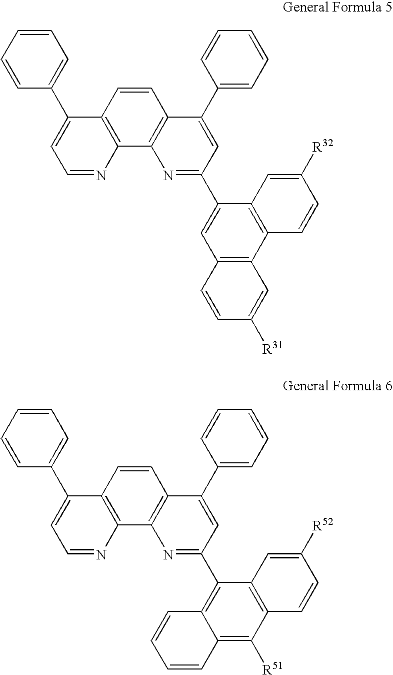

Note in General Formula 5 or 6 that R³¹, R³², R⁵¹, and R⁵² each represent hydrogen, an alkyl group, or an aryl group.

The following are specific examples for compounds of General Formulas 2 to 6. First, as specific examples for the compound of General Formula 2, there are 2,4,7-triphenyl-1,10-phenanthroline (Structural Formula 11), 2-(4-methylphenyl)-4,7-diphenyl-1,10-phenanthroline (Structural Formula 12), 4,7-diphenyl-2-(2,4,6-trimethylphenyl)-1,10-phenanthroline (Structural Formula 13), 2-(4-t-butylphenyl)-4,7-diphenyl-1,10-phenanthroline (Structural Formula 14), 2-(4-i-propylphenyl)-4,7-diphenyl-1,10-phenanthroline (Structural Formula 15), 2-(4-cyclohexylphenyl)-4,7-diphenyl-1,10-phenanthroline (Structural Formula 16), and the like, and the structural formulas are shown in Structural Formulas 11 to 16.

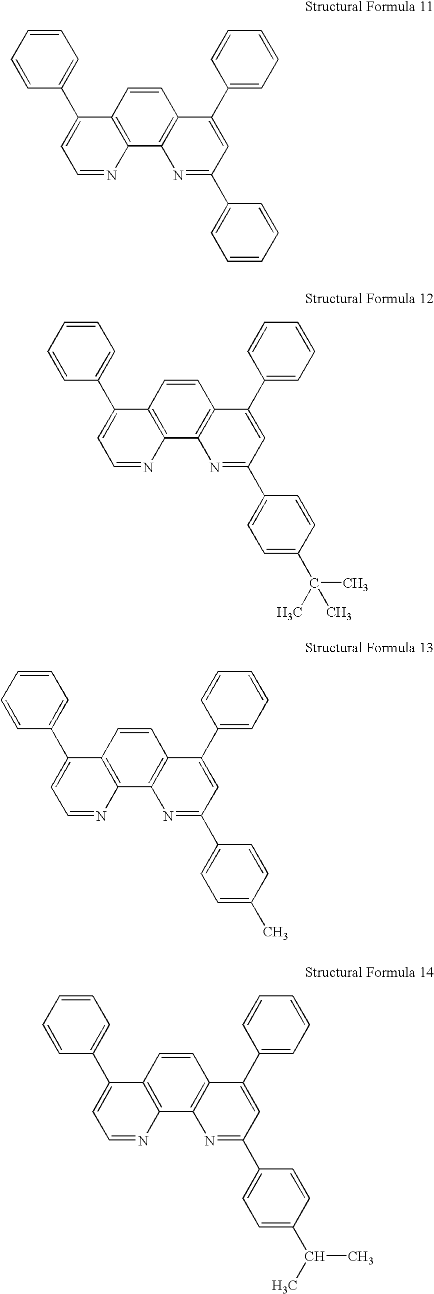

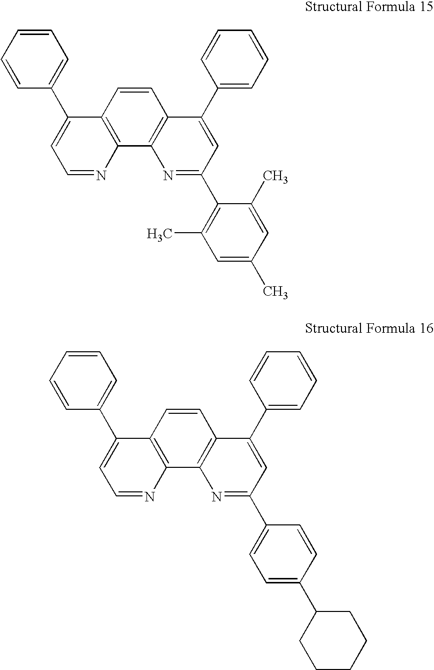

Further, as specific examples for the compound of General Formula 2, there are 2-(4-phenylphenyl)-4,7-diphenyl-1,10-phenanthroline (Structural Formula 17), 2-(2-phenylphenyl)-4,7-diphenyl-1,10-phenanthroline (Structural Formula 18), 2-(3,5-diphenylphenyl)-4,7-diphenyl-1,10-phenanthroline (Structural Formula 19), and the like, and the structural formulas are shown in Structural Formulas 17 to 19.

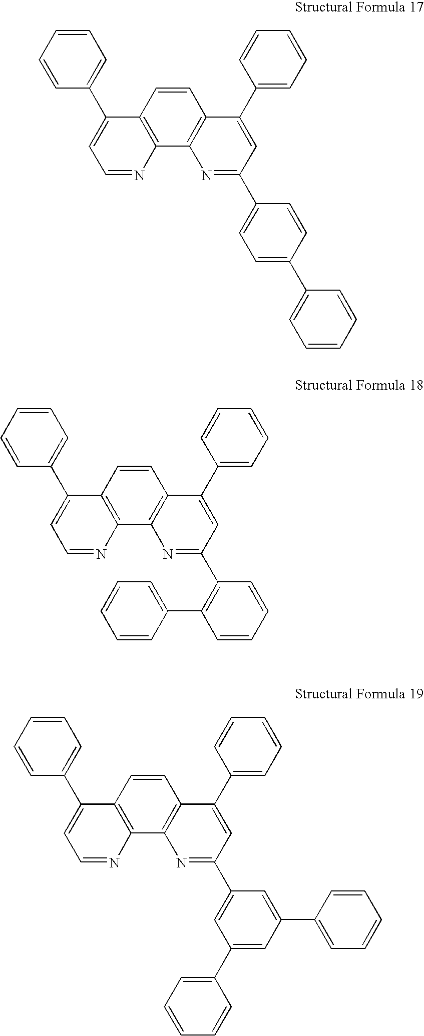

Next, as specific examples for the compound of General Formula 3, there are 2-(2-naphthyl)-4,7-diphenyl-1,10-phenanthroline (Structural Formula 20), 2-[2-(6-t-butyl)naphthyl]-4,7-diphenyl-1,10-phenanthroline (Structural Formula 21), 2-[2-(6-methyl)naphthyl]-4,7-diphenyl-1,10-phenanthroline (Structural Formula 22), 2-[2-(6-phenyl)naphthyl]-4,7-diphenyl-1,10-phenanthroline (Structural Formula 23), and the like, and the structural formulas are shown in Structural Formulas 20 to 23.

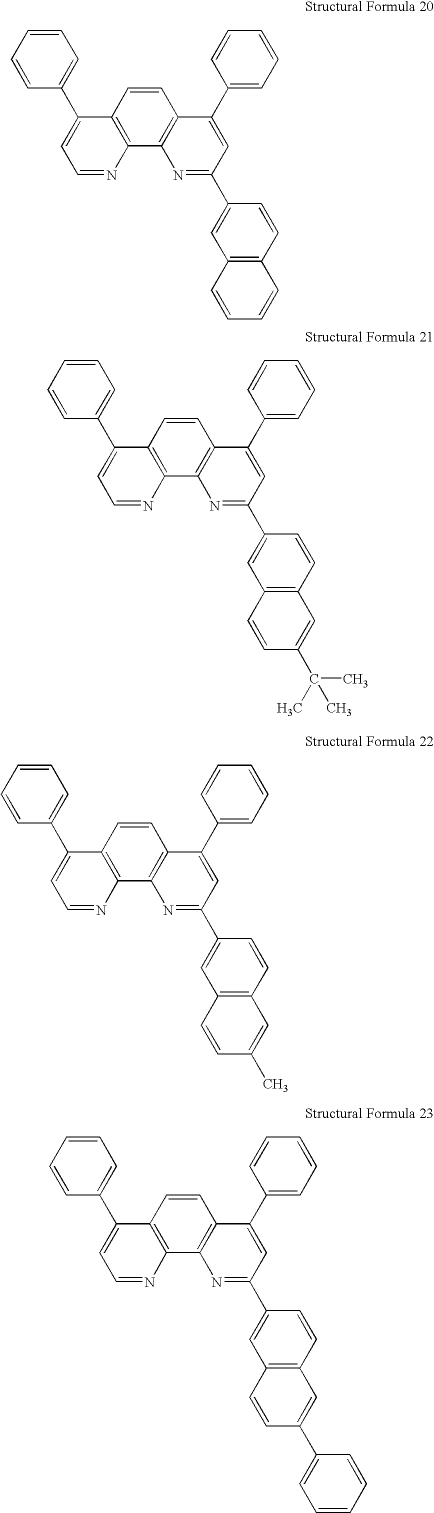

Next, as specific examples for the compound of General Formula 4, there are 2-(1-naphthyl)-4,7-diphenyl-1,10-phenanthroline (Structural Formula 24), 2-[1-(4-t-butyl)naphthyl]-4,7-diphenyl-1,10-phenanthroline (Structural Formula 25), 2-[1-(5-t-butyl)naphthyl]-4,7-diphenyl-1,10-phenanthroline (Structural Formula 26), 2-[1-(4-methyl)naphthyl]-4,7-diphenyl-1,10-phenanthroline (Structural Formula 27), 2-[1-(5-methyl)naphthyl]-4,7-diphenyl-1,10-phenanthroline (Structural Formula 28), 2-[1-(5-phenyl)naphthyl]-4,7-diphenyl-1,10-phenanthroline (Structural Formula 29), and the like, and the structural formulas are shown in Structural Formulas 24 to 29.

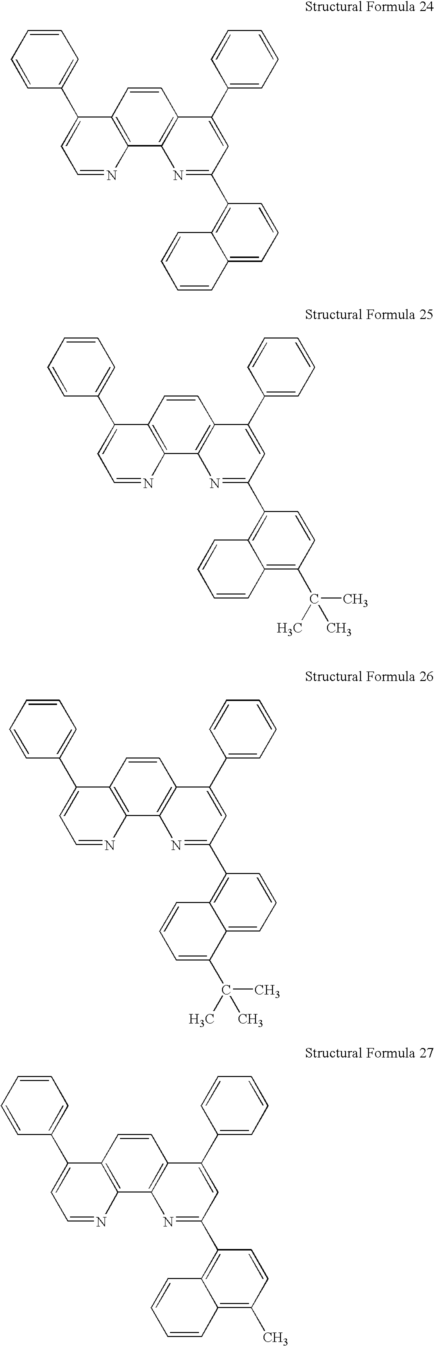

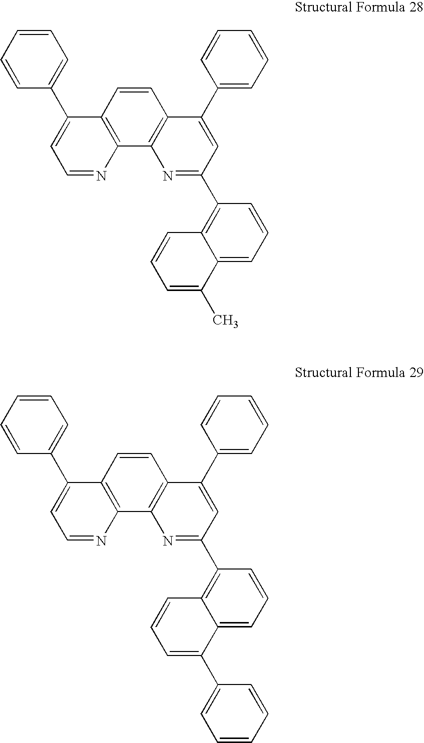

Further, as specific examples for the compound of General Formula 5, there are 2-(9-phenanthryl)-4,7-diphenyl-1,10-phenanthroline (Structural Formula 30), 2-(9-(3-t-butyl)phenanthryl-4,7-diphenyl-1,10-phenanthroline (Structural Formula 31), 2-[9-(3-methyl)phenanthryl]-4,7-diphenyl-1,10-phenanthroline (Structural Formula 32), 2-[9-(7-t-butyl)phenanthryl]-4,7-diphenyl-1,10-phenanthroline (Structural Formula 33), 2-[9-(3-phenyl)phenanthryl]-4,7-diphenyl-1,10-phenanthroline (Structural Formula 34), 2-[9-(7-phenyl)phenanthryl]-4,7-diphenyl-1,10-phenanthroline (Structural Formula 35), 2-[9-(7-methyl)phenanthryl]-4,7-diphenyl-1,10-phenanthroline (Structural Formula 36), and the like, and the structural formulas are shown in Structural Formulas 30 to 36.

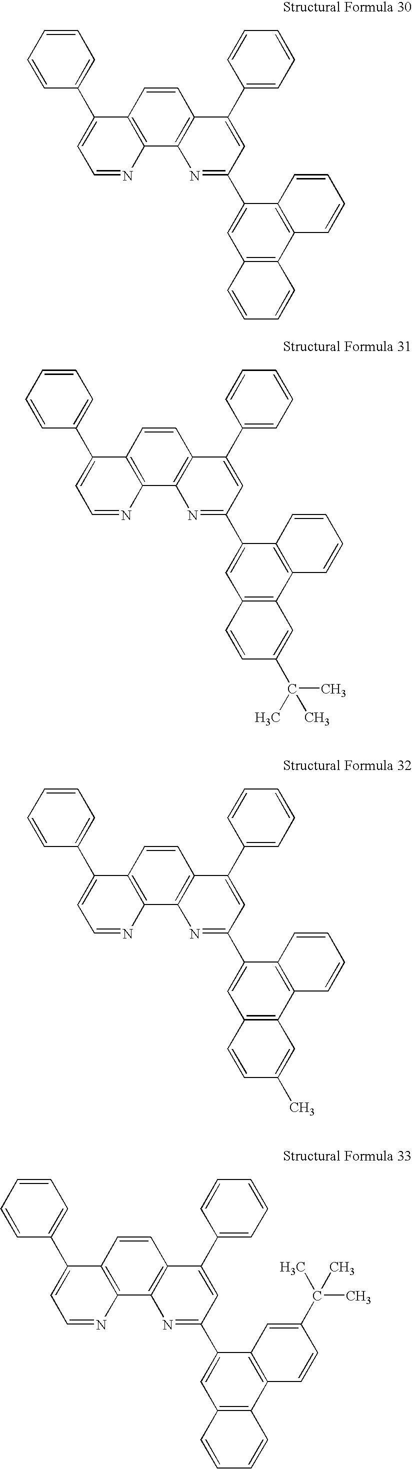

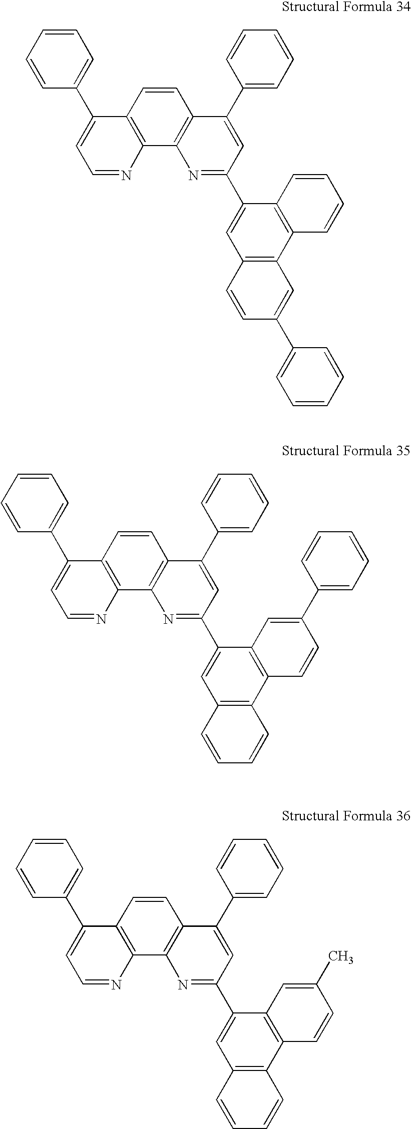

Last, as specific examples for the compound of General Formula 6, there are 2-(9-anthryl)-4,7-diphenyl-1,10-phenanthroline (Structural Formula 37), 2-[9-(2-t-butyl)anthryl]-4,7-diphenyl-1,10-phenanthroline (Structural Formula 38), 2-[9-(2-methyl)anthryl]-4,7-diphenyl-1,10-phenanthroline (Structural Formula 39), 2-[9-(10-methyl)anthryl]-4,7-diphenyl-1,10-phenanthroline (Structural Formula 40), 2-[9-(10-phenyl)anthryl]-4,7-diphenyl-1,10-phenanthroline (Structural Formula 41), and the like, and the structural formulas are shown in Structural Formulas 37 to 41.

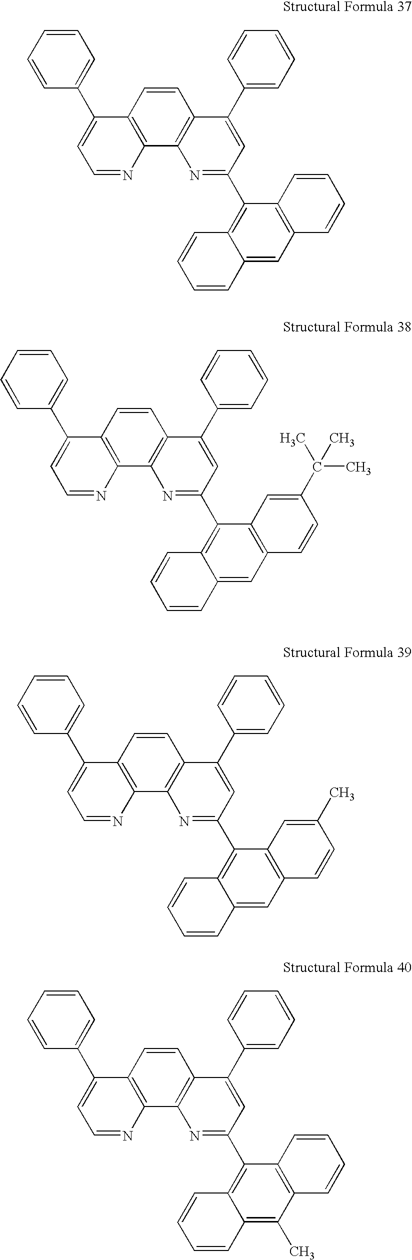

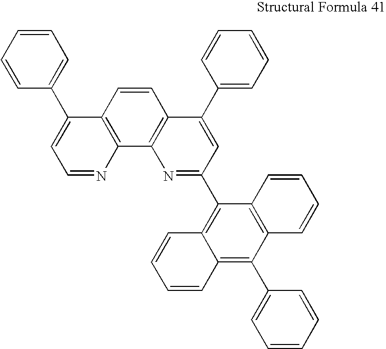

### EMBODIMENT MODE 2

Next to the embodiment mode of a phenanthroline derivative compound of the present invention, an embodiment mode for a manufacturing method thereof will be described in detail. A phenanthroline derivative compound of the present invention can be manufactured by the following synthetic reaction. First, aryl halide capable of forming an appropriate aryl group is reacted with Mg, n-BuLi (n-butyllithium), or t-BuLi (t-butyllithium) to form a corresponding organic metal compound.

This organic metal compound is reacted with 4,7-diphenyl-1,10-phenanthroline that has phenyl groups at the fourth and seventh positions, and is then reacted with a proton source such as water or alcohol to generate a 1:1 adduct which is an intermediate. Subsequently, the generated 1:1 adduct is oxidized by MnO₂ in a solvent such as CH₂Cl₂ (dichloromethane), which is stable against oxidation, so as to eliminate hydrogen bonded with nitrogen at the first position and hydrogen bonded with carbon at the second position; accordingly, the phenanthroline derivative compound of the present invention is fabricated. The reaction process can be expressed by the following Reaction Formula 1.

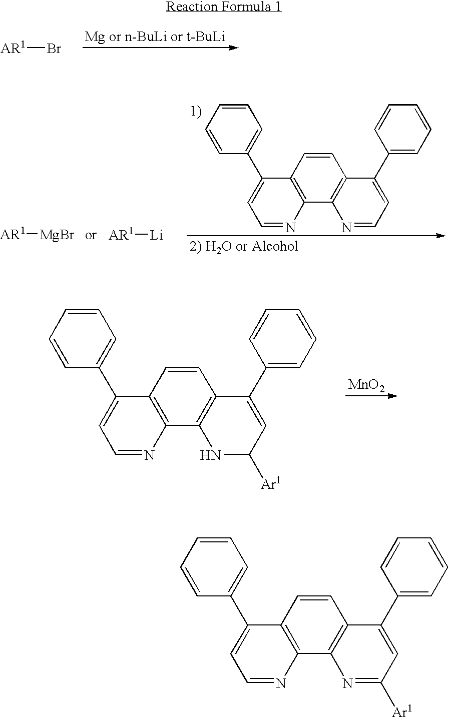

The reaction condition of the fabrication is not particularly limited as long as an intermediate and a final product can be both formed. As for a pressure for example, the reaction can be conducted under from reduced pressure to increased pressure but preferably conducted under atmospheric pressure in terms of safety and operability. A reaction temperature is not particularly limited as well, but the reaction is preferably conducted under reflux in terms of a reaction rate. A reaction tool (structure) to be used is not particularly limited, and various reaction tools can be used, which can be exemplified by an eggplant-type flask, a round flask, a conical flask, or the like. Further, a material of the reaction tool is not particularly limited, and various kinds of materials can be used, which can be exemplified by glass, stainless steel, ceramic, and the like.

As already mentioned above, the phenanthroline derivative compound of the present invention manufactured in the above-described manner, has an excellent electron transporting property and an excellent property of hardly causing crystallization, so that life of the element can be lengthened. Therefore, the phenanthroline derivative compound of the present invention can be preferably utilized for an electron transporting material or an organic EL element. Further, when being used as a light emitting element, since the phenanthroline derivative compound has a phenanthroline skeleton and has a wide band gap, it can exhibit an excellent characteristic as an electron transporting layer or a hole blocking layer.

### EMBODIMENT MODE 3

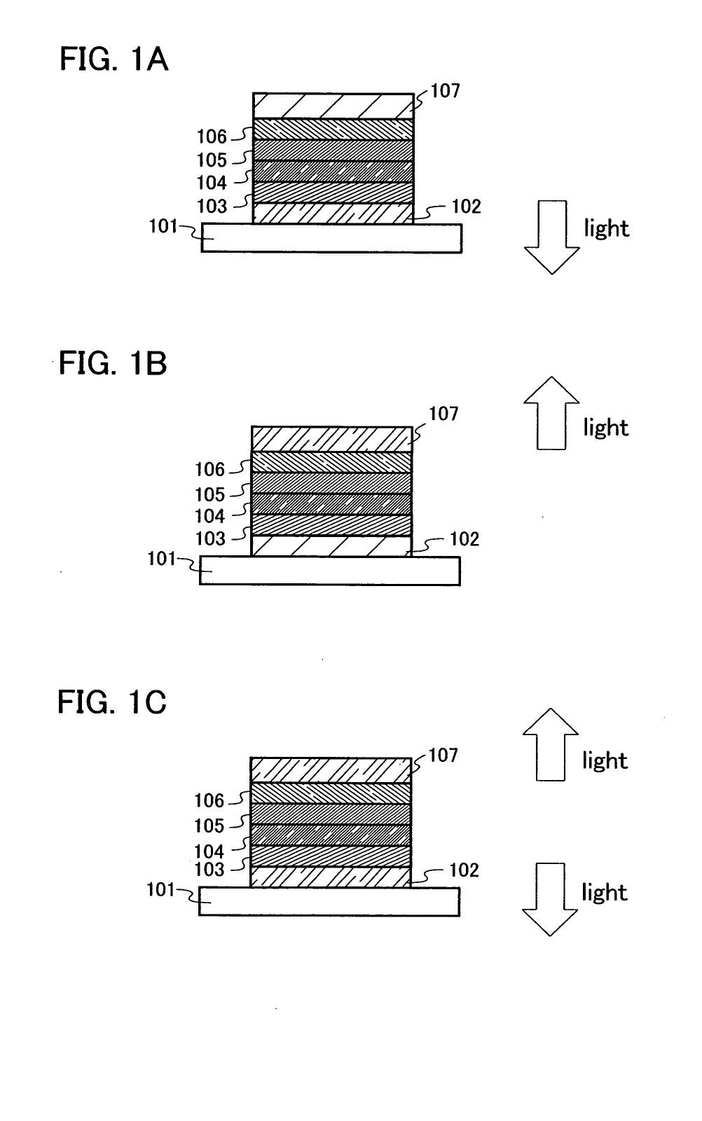

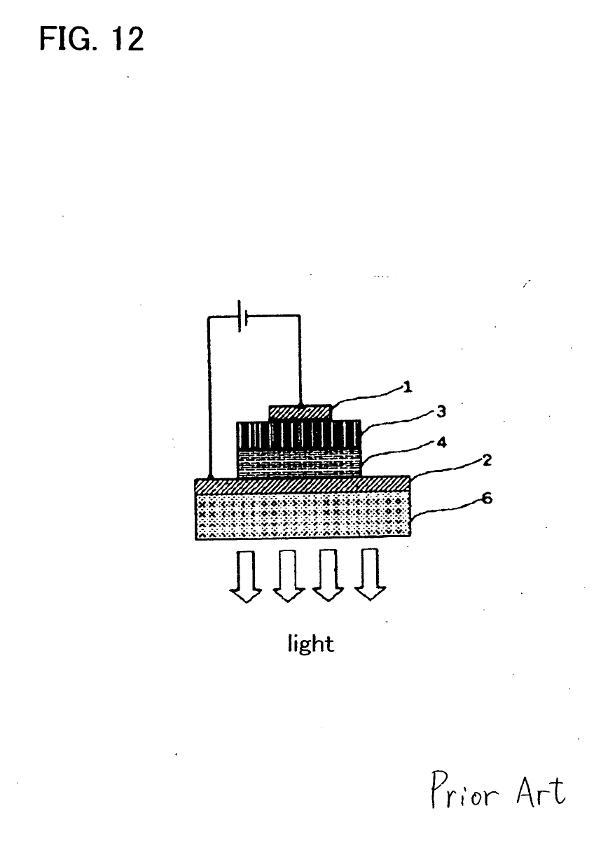

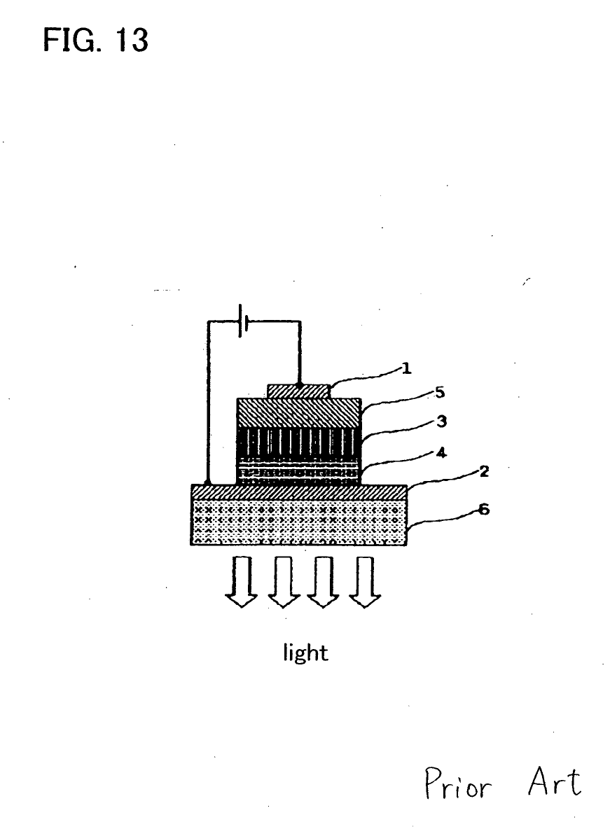

Next, an embodiment mode of a light emitting element of the present invention will be described in detail. A phenanthroline derivative compound of the present invention can be used in a similar structure to that of a conventional light emitting element having a single-layer structure shown in FIG. 12; however, in the light emitting element of the present invention, a stacked structure having a plurality of layers stacked between a pair of electrodes as shown in FIGS. 1A to 3 is preferably employed. The plurality of layers are stacked layers formed by combining layers containing a substance having a high carrier injecting property or a high carrier transporting property, so that a light emitting region is formed in a place which is away from the electrodes, that is, so as to perform reconbination of carriers in a portion which is away from the electrodes. First, a mode of the light emitting element is described with reference to FIG. 1A.

In the modes of FIGS. 1A to 1C, the light emitting element includes a first electrode 102; a first layer 103, a second layer 104, a third layer 105, and a forth layer 106 which are sequentially stacked over the first electrode 102; and a second electrode 107 further provided thereover. The following description is made of the condition that the first electrode 102 of FIGS. 1A to 1C serves as an anode and the second electrode 107 serves as a cathode. A substrate 101 is used as a support medium of the light emitting element, and as the substrate 101, glass, plastic, or the like can be used for example. Note that another material may be used as long as the material functions as a support medium in the manufacturing process of the light emitting element.

As the first electrode 102, a metal, an alloy, a conductive compound, a mixture thereof, or the like having a high work function (specifically, of 4.0 eV or more) is preferably used. Specifically, indium tin oxide (ITO), indium tin oxide containing silicon, indium zinc oxide (IZO) in which indium oxide is mixed with 2 to 20 wt % of zinc oxide (ZnO), indium oxide (IWZO) containing 0.5 to 5 wt % of tungsten oxide and 0.1 to 1 wt % of zinc oxide, or the like can be used, for example.

Such a conductive metal oxide film is usually formed by a sputtering method, but may be formed by applying a sol-gel method or the like. Further, gold (Au), platinum (Pt), nickel (Ni), tungsten (W), chromium (Cr), molybdenum (Mo), iron (Fe), cobalt (Co), copper (Cu), palladium (Pd), a nitride of a metal material (for example, titanium nitride: TiN), or the like can be used.

The first layer 103 is a layer containing a high hole injecting property, and molybdenum oxide (MoOₓ), vanadium oxide (VOₓ), ruthenium oxide (RuOₓ), tungsten oxide (WOₓ), manganese oxide (MnOₓ), or the like can be used. Alternatively, the first layer 103 can be formed by using a phthalocyanine-based compound such as phthalocyanine (H₂Pc) or copper phthalocyanine (CuPc), a high molecular material such as poly(ethylene dioxythiophene)/poly(styrenesulfonic acid) (PEDOT/PSS), or the like.

A composite material containing an organic compound and an inorganic compound may also be used for the first layer 103. In particular, a composite material containing an organic compound and an inorganic compound which has an electron accepting property to the organic compound is superior in a hole injecting property and a hole transporting property, because electrons are transferred between the organic compound and the inorganic compound to increase a carrier density. In this case, it is preferable to use a substance having a superior hole transporting property as the organic compound. Specifically, an aromatic amine-based organic compound or a carbazole-based organic compound is preferable.

As the inorganic compound, a substance having an electron accepting property to the organic compound is preferable, and an oxide of a transition metal is specifically preferable. For example, a metal oxide such as titanium oxide (TiOₓ), vanadium oxide (VOₓ), molybdenum oxide (MoOₓ), tungsten oxide (WOₓ), rhenium oxide (ReOₓ), ruthenium oxide (RuOₓ), chromium oxide (CrOₓ), zirconium oxide (XrOₓ), hafnium oxide (HfOₓ), tantalum oxide (TaOₓ), silver oxide (AgOₓ), or manganese oxide (MnOₓ) can be used. In the case of using the composite material containing an organic compound and an inorganic compound for the first layer 103, an ohmic contact with the first electrode 102 can be made; therefore, the material for the first electrode 102 can be selected regardless of a work function.

As a substance for forming the second layer 104, a substance having a high hole transporting property, specifically, an aromatic amine-based (that is, a material having a benzene ring-nitrogen bond) compound is preferable. As the substance for the second layer 104, the following substances can be used, which is a substance having a hole mobility of 10⁻⁶ cm²/Vs or more mainly. However, other substances may be used if the substances have a hole transporting property higher than an electron transporting property. As the second layer 104, a mixed layer of the above-described substances or a stacked layer containing two or more layers may be employed as well as a single layer.

As the material which is widely used, a starburst aromatic amine compound such as 4,4′-bis[N-(3-methylphenyl)-N-phenylamino]-biphenyl; a derivative thereof: 4,4′-bis[N-(1-naphthyl)-N-phenylamino]biphenyl (hereinafter abbreviated as NPB); 4,4′, 4″-tris(N,N-diphenyl-amino)triphenylamine; or 4,4′, 4″-tris[N-(3-methylphenyl)-N-phenylamino]triphenylamine can be used.

The third layer 105 has a layer containing a light emitting substance, and a phenanthroline derivative compound of the present invention can be used together with this light emitting substance to form a light emitting element. In that case, a superior property can be exhibited by utilizing the phenanthroline derivative compound of the present invention as a host of the light emitting layer. The light emitting substance in using the phenanthroline derivative compound of the present invention to the light emitting layer is not particularly limited and various kinds can be used.

As the light emitting substance, the following can be used: a coumarin derivative such as coumarin 6 or coumarin 545T; a quinacridone derivative such as N,N′-dimethyl quinacridone or N,N′-diphenyl quinacridone; an acridone derivative such as N-phenylacridone or N-methyl acridone; a condensed aromatic compound such as 2-t-butyl-9,10-di(2-naphthyl)anthracene (t-BuDNA), 9,10-diphenylanthracene, 2,5,8,11-tetra-t-butylperylene, or rubrene; a pyran derivative such as 4-dicyanomethylene-2-[p-(dimethylamino)styryl]6-methyl-4H-pyran, an amine derivative such as 4-(2,2-diphenylvinyl)triphenylamine, and the like.

The above-described light emitting substance emit fluorescence; however a phosphorescent substance which emits phosphorescence can be also used in the present invention, which can be an iridium complex such as bis{2-(ρ-tolyl)pyridinato}(acetylacetonato)iridium(III), bis{2-(2′-benzothienyl)pyridinato}(acetylacetonato)iridium(III), or bis{2-(4,6-difluorophenyl)pyridinato]picolinatoiridium(III); a platinum complex such as 2,3,7,8,12,13,17,18-octaethyl-21H,23H-porphyrin-platinum complex, a rare-earth complex such as 4,7-diphenyl-1,10-phenanthroline-tris(2-thiophenyltrifluoroacetonato)europium(III), and the like.

When the light emitting substance is used, a second host material such as CBP can also be used together, and the second host material used together is not particularly limited and various kinds can be used, which can be CBP, 4,4′-bis[N-(1-naphthyl)-N-phenylamino]biphenyl (NPB), tris(8-quinolinolato)aluminum (Alq), and the like.

A substance having a high electron transporting property can be preferably used as a substance for forming the fourth layer 106. As the substance having a high electron transporting property, a metal complex can be used, such as tris(8-quinolinolato)aluminum (Alq), tris(4-methyl-8-quinolinolato)aluminum (Almq₃), bis(2-methyl-8-quinolinolato)(4-phenylphenolato)aluminum (BAlq), tris(8-quinolinolato)gallium (Gaq₃), bis(2-methyl-8-quinolinolato)(4-phenylphenolato)gallium (BGaq), bis(10-hydroxybenzo[h]quinolinato)beryllium (BeBq₂), bis[2-(2′-hydroxyphenyl)benzoxazolato]zinc (Zn(BOX)₂), bis[2-(2′-hydroxyphenyl)benzothiazolato]zinc (Zn(BTZ)2), or the like.

Other than the metal complex, 2-(4-biphenylyl)-5-(4-tert-buthylphenyl)-1,3,4-oxadiazole (PBD), 1,3-bis[5-(p-tert-buthylphenyl)-1,3,4-oxadiazol-2-yl]benzene (OXD-7), 3-(4-tert-butylphenyl)-4-phenyl-5-(4-biphenylyl)-1,2,4-triazole (TAZ), 3-(4-tert-butylphenyl)-4-(4-ethylphenyl)-5-(4-biphenylyl)-1,2,4-triazole (p-EtTAZ), bathophenanthroline (BPhen), bathocuproin (BCP), or the like can be employed.

In the case of using the phenanthroline derivative compound of the present invention as an electron transporting material, since it has a superior electron transporting property, it can be preferably used for the fourth layer 106. The fourth layer 106 may be not only a single layer but also a mixed layer or a stacked layer of two or more layers. For example, a stacked layer having two or more layers of a layer containing a phenanthroline derivative compound of the present invention and a layer containing an electron transporting material may be used.

As a substance for forming the second electrode 107, a metal, an alloy, a conductive compound, a mixture thereof, or the like having a low work function (of 3.8 eV or less) can be used. As a specific example of such a cathode material, an element belonging to group 1 or 2 in the periodic table, that is, an alkali metal such as lithium (Li) or cesium (Cs) or an alkaline earth metal such as magnesium (Mg), calcium (Ca), or strontium (Sr); an alloy containing the element belonging to group 1 or 2 (MgAg, AlLi); a rare-earth metal such as europium (Eu) or ytterbium (Yb); an alloy thereof; or the like can be used.

The substance for forming the second electrode 107 is not limited to those described above, and various conductive materials such as Al, Ag, ITO, and ITO containing silicon, regardless of the work function, can be used for the second electrode 107, by providing a layer having a function of promoting electron injection between the second electrode 107 and the fourth layer 106. For the layer having a function of promoting electron injection, a compound of an alkali metal or an alkaline earth metal such as lithium fluoride (LiF), cesium fluoride (CsF), or calcium fluoride (CaF₂) can be used.

Further alternatively, a layer containing a substance having an electron transporting property into which an alkali metal or an alkaline earth metal is added can be used as the layer having a function of promoting electron injection. As the substance having an electron transporting property, the above-described substance having a high electron transporting property can be used. For the layer having a function of promoting electron injection, Alq into which magnesium (Mg) or lithium (Li) is added, the phenanthroline derivative compound of the present invention into which magnesium (Mg) or lithium (Li) is added, or the like can be used.

In forming the above-described each layer as well as the electrodes, various film forming methods can be employed without any particular limitation, which are exemplified by an inkjet method, a printing method, an evaporation method, a spin coating method, and the like. It is to be noted that differing film forming methods can be used for each layers and the electrodes.

A current flows due to a potential difference generated between the first electrode 102 and the second electrode 107, and holes and electrons are recombined in the third layer 105 which is a layer containing a light emitting substance; accordingly, the light emitting element of the present invention having the above-described structure emits light. In other words, a structure where a light emitting region is formed in the third layer 105 is obtained. The light is extracted through either or both of the first electrode 102 and the second electrode 107. Therefore, either or both of the first electrode 102 and the second electrode 107 is formed with a light-transmitting substance.

In the case where only the first electrode 102 is formed with a light-transmitting substance, light is extracted from a substrate side through the first electrode 102, as shown in FIG. 1A. On the contrary, in the case where only the second electrode 107 is formed with a light-transmitting substance, light is extracted from an opposite side to the substrate through the second electrode 107, as shown in FIG. 1B. Further, when both of the first electrode 102 and the second electrode 107 are formed with light-transmitting substances, light is extracted from both the substrate side and the opposite side to the substrate through the first electrode 102 and the second electrode 107, as shown in FIG. 1C.

Note that the structure of layers provided between the first electrode 102 and the second electrode 107 is not limited to the above-described structure. Other structures may be employed as long as a light emitting region for recombining holes and electrons is provided in a place which is away from the first electrode 102 and the second electrode 107 for the purpose of suppressing quenching caused by adjacence of a light emitting region and a metal.

In other words, in the light emitting element of the present invention, the stacked structure of layers is not particularly limited, and a layer containing a substance having a high electron transporting property, a substance having a high hole transporting property, a substance having a high electron injecting property, a substance having a high hole injecting property, a bipolar substance (a substance having high electron or hole transporting property), a hole blocking material, or the like, may be freely combined with a layer containing a phenanthroline derivative compound of the present invention. For example, as shown in FIG. 3, a hole blocking layer 108 may be provided between the third layer 105 and the forth layer 106. Since a phenanthroline derivative of the present invention has a wide band gap, it can be used as a hole blocking material.

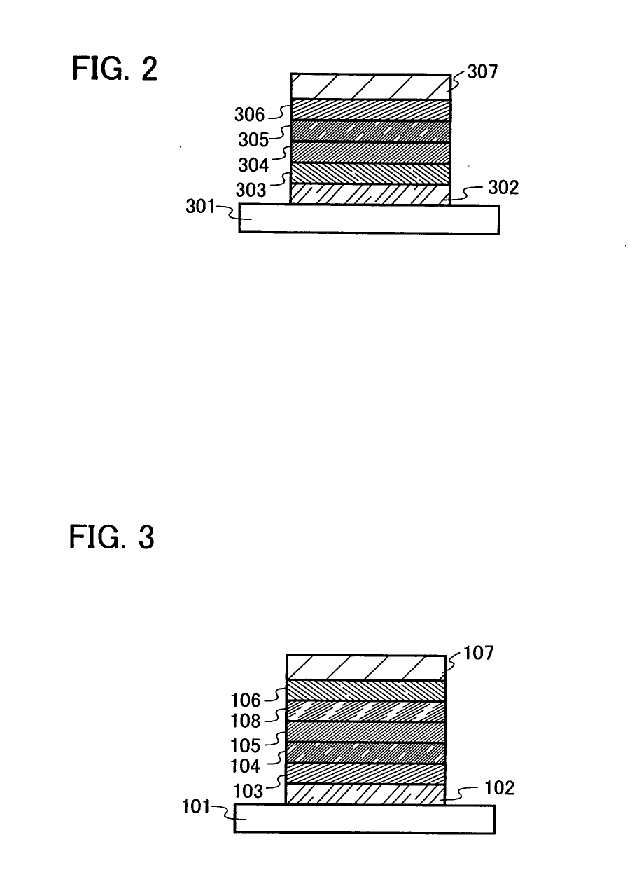

A light emitting element shown in FIG. 2 has a structure in which a first layer 303 containing a substance having a high electron transporting property, a second layer 304 containing a light emitting substance, a third layer 305 containing a substance having a high hole transporting property, a fourth layer 306 containing a substance having a high hole injecting property, and a second electrode 307 serving as an anode, are stacked in order over a first electrode 302 serving as a cathode. Note herein that reference numeral 301 denotes a substrate.

In the present embodiment mode shown in FIGS. 1A to 3, the light emitting element is manufactured over a substrate formed of glass, plastic, or the like. By manufacturing a plurality of light emitting elements over one substrate, passive light emitting elements can be manufactured. In addition, a thin film transistor (TFT) may be, for example, formed over a substrate containing glass, plastic, or the like, and a light emitting element may be manufactured over an electrode electrically connected to the TFT.

Thus, an active-matrix light emitting device which controls driving of the light emitting element by using the TFT can be manufactured. The structure of a TFT is not particularly limited in the light emitting device of the present invention. A staggered TFT or an inversely staggered TFT may be used. A driver circuit formed over a TFT array substrate may be formed with either or both of an n-type TFT and a p-type TFT.

### EMBODIMENT MODE 4

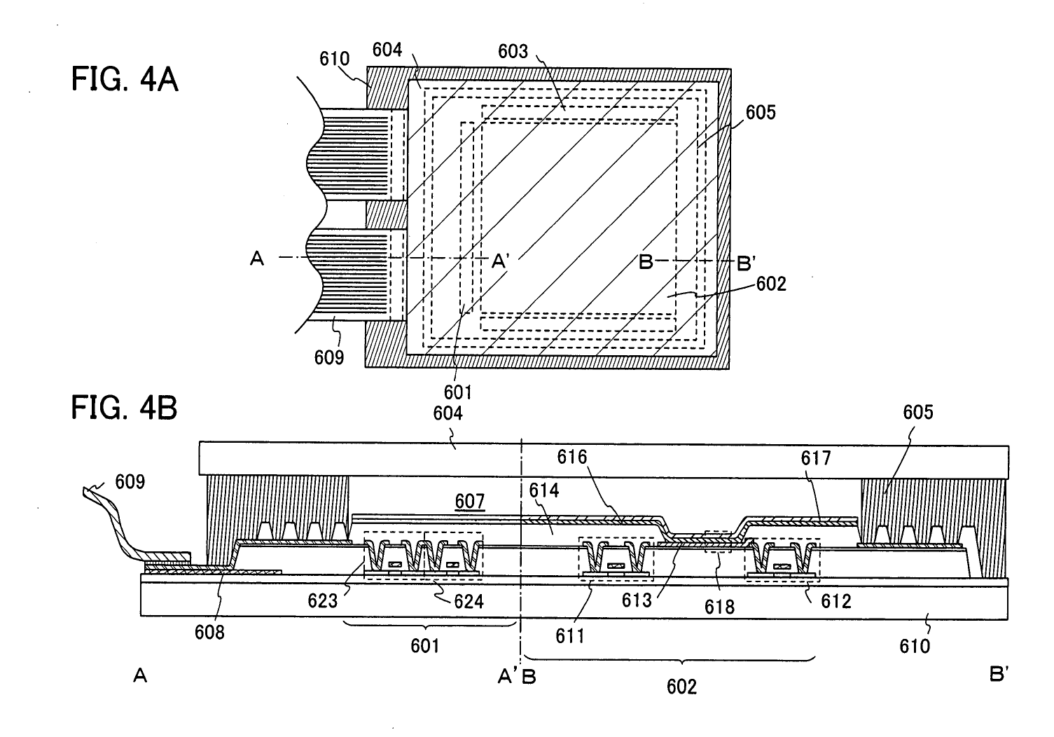

Next to the embodiment mode of the light emitting element, Embodiment Mode 4 describes a light emitting device having a light emitting element of the present invention. A light emitting device having a light emitting element of the invention in a pixel portion will be described in this embodiment mode with reference to FIGS. 4A and 4B. FIG. 4A is a top view showing the light emitting device and FIG. 4B is a cross-sectional view taken along line A-A′ and B-B′ of FIG. 4A. Reference numeral 601 indicated by dotted lines denotes a driver circuit area (a source driver circuit); 602, a pixel portion; and 603, a driver circuit area (a gate driver circuit). Reference numeral 604 denotes a sealing substrate; and 605, a sealant; and a portion surrounded by the sealant 605 is a space 607.

In FIGS. 4A and 4B, a lead wire 608 is a wire for transmitting a signal to be input to the source driver circuit 601 and the gate driver circuit 603 and receives a video signal, a clock signal, a start signal, a reset signal, or the like from an FPC (flexible printed circuit) 609 that is an external input terminal. Note that only the FPC is shown here; however, the FPC may be provided with a printed wiring board (PWB). The light emitting device in this specification means not only a light emitting device itself but also a light emitting device to which an FPC or a PWB is attached.

Subsequently, a cross-sectional structure is described with reference to FIG. 4B. The driver circuit area and the pixel portion are formed over an element substrate 610. Here, the source driver circuit 601 which is the driver circuit area and one pixel in the pixel portion 602 are shown. A CMOS circuit which is a combination of an n-channel TFT 623 and a p-channel TFT 624 is formed as the source driver circuit 601; however, a TFT for forming the driver circuit may be formed using the CMOS circuit, a PMOS circuit, or an NMOS circuit. A driver integration type in which a driver circuit is formed over a substrate is described in this embodiment mode, but it is not necessarily required and a driver circuit can be formed outside the substrate.

The pixel portion 602 has a plurality of pixels, each of which includes a switching TFT 611, a current control TFT 612, and a first electrode 613 which is electrically connected to a drain of the current control TFT 612. An insulator 614 is formed to cover an end of the first electrode 613. Either a negative type resin that is insoluble in an etchant due to light or a positive type resin that is dissoluble in an etchant due to light can be used as the insulator 614. Here, a positive type photosensitive acrylic resin film is used as the insulator 614.

In order to favorably perform film formation, the insulator 614 is formed to have a curved surface at an upper end or a lower end thereof. For example, in the case of using a positive type photosensitive acrylic as a material of the insulator 614, the insulator 614 is preferably formed to have a curved surface with a curvature radius (of 0.2 μm to 3 μm) only at an upper end.

A layer 616 containing a light emitting substance and a second electrode 617 are formed over the first electrode 613. In this embodiment mode, a material having a high work function is preferably used as a material for the first electrode 613 which serves as an anode. For example, the first electrode 613 can be formed by using a single-layer film such as an ITO film, an indium tin oxide film containing silicon, an indium oxide film containing zinc oxide of 2 to 20 wt %, a titanium nitride film, a chromium film, a tungsten film, a Zn film, or a Pt film; a stacked layer of a titanium nitride film and a film containing aluminum as its main component; a three-layered structure of a titanium nitride film, a film containing aluminum as its main component, and another titanium nitride film; or the like. When the first electrode 613 has a stacked structure, it can have low resistance as a wire and form a favorable ohmic contact.

The layer 616 containing a light emitting substance contains the phenanthroline derivative compound of the present invention, described in Embodiment Mode 1. The phenanthroline derivative compound of the present invention has a superior electron transporting property and hardly causes crystallization. Accordingly, by using a layer containing the phenanthroline derivative compound of the present invention, a long-life light emitting device can be obtained. Further, various materials can be used as a material used in combination with the phenanthroline derivative compound of the present invention described in Embodiment Mode 1, and a low molecular material, a medium molecular material (including oligomer and dendrimer), or a high molecular material may be used.

As a material for the second electrode (cathode) 617 which is formed over the layer 616 containing a light emitting substance, a material having a low work function (Al, Ag, Li, or Ca; an ally thereof such as MgAg, MgIn, or AlLi; or a compound thereof such as CaF₂, Li₂O, or LiF) is preferably used. In the case where light generated in the layer 616 containing a light emitting substance is made to pass through the second electrode 617, stacked films of a metal thin film a thickness of which is reduced and a transparent conductive film (ITO, indium oxide containing zinc oxide of 2 to 20 wt %, indium tin oxide containing silicon, zinc oxide (ZnO), or the like) are preferably used.

Further in this embodiment mode, the sealing substrate 604 and the element substrate 610 are attached by the sealant 605, thereby forming a structure where a light emitting element 618 is provided in the space 607 surrounded by the element substrate 610, the sealing substrate 604, and the sealant 605. The space 607 may be filled with an inert gas (such as nitrogen or argon) or the sealant 605, and various kinds of materials can be used as the material to fill the space 607 without any particular limitation.

The sealant 605 is preferably a material which does not transmit moisture and oxygen as much as possible, and an epoxy resin is preferably used. As the sealing substrate 604, a glass substrate, a quartz substrate, or a plastic substrate formed of FRP (Fiberglass-Reinforced Plastics), PVF (polyvinyl fluoride), mylar, polyester, acrylic, or the like can be used. In the above-described manner, a light emitting device having a light emitting element of the present invention can be obtained.

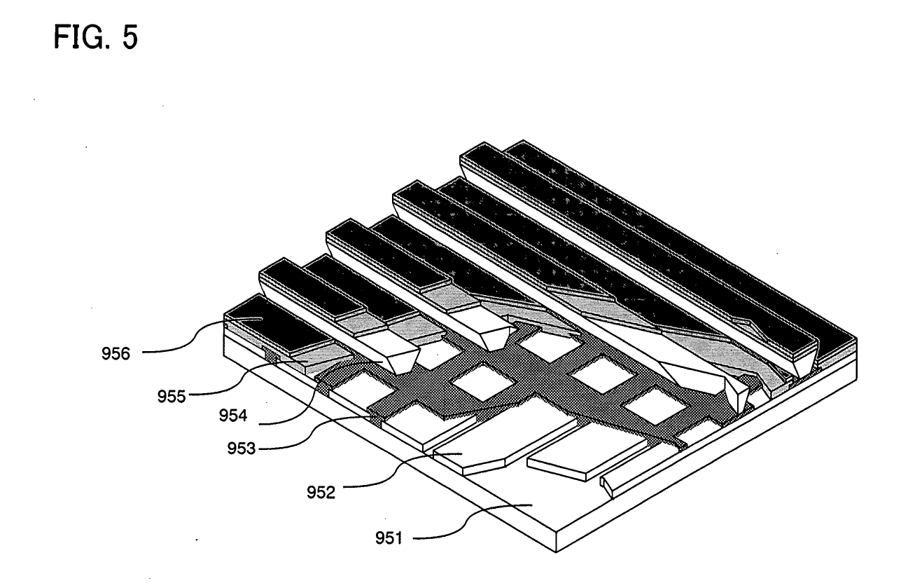

In this embodiment mode, an active light emitting device which controls driving of a light emitting element by a transistor is described as a light emitting device of the present invention. Alternatively, a passive light emitting device which drives a light emitting element without providing an element for driving such as a transistor may be used. FIG. 5 is a perspective view of a passive light emitting device manufactured in accordance with the present invention. In FIG. 5, a layer 955 containing a light emitting substance is provided between an electrode 952 and an electrode 956, over a substrate 951. An end portion of the electrode 952 is covered by an insulating layer 953.

Over the insulating layer 953, a partition wall layer 954 is provided. The partition wall layer 954 has a slope shape such that the distance between one sidewall and other side of the partition wall layer 954 is reduced toward a surface of the substrate. In other words, a cross section of the partition wall layer 954 in a narrow side direction shows a trapezoid shape having a shorter base (a side parallel to a surface of the insulating layer 953 and contacting with the insulating layer 953) than an upper side (a side parallel to a surface of the insulating layer 953 and not contacting with the insulating layer 953). By providing the partition wall layer 954 in this manner, a defect of a light emitting element due to static electricity or the like can be prevented. A passive light emitting device can be also driven with small power consumption by including a light emitting element of the present invention which operates with low driving voltage.

### EMBODIMENT MODE 5

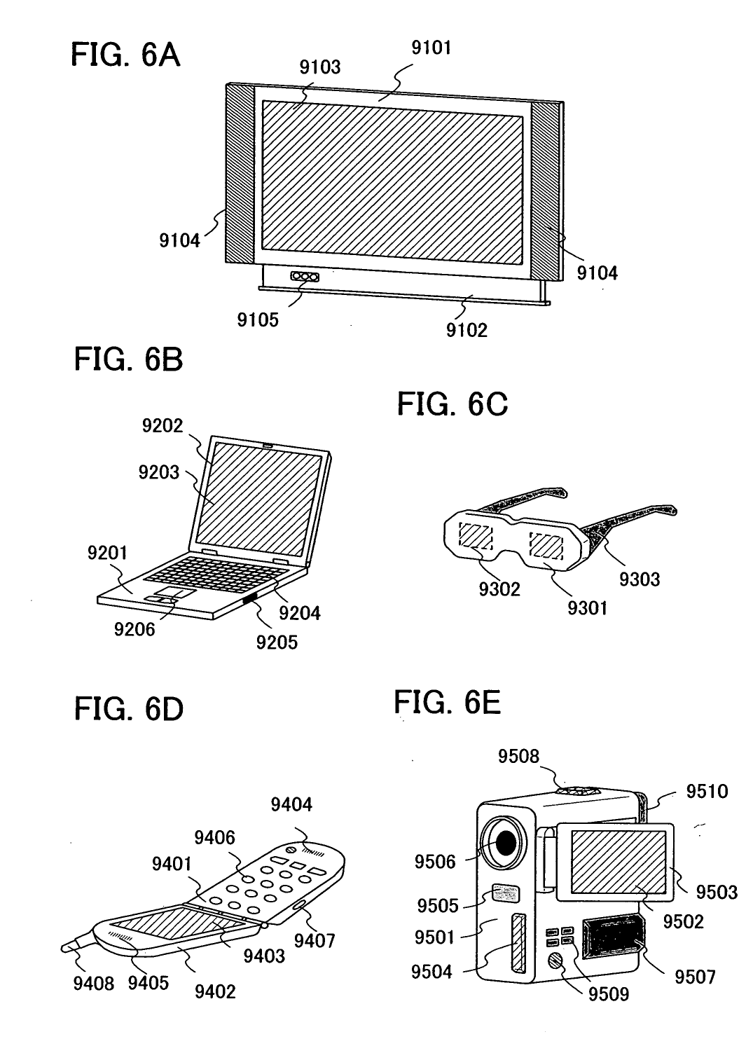

Last, various electronic devices of the present invention provided with a light emitting device using a light emitting element of the present invention will be described in this embodiment mode. As examples of electronic devices of the present invention using the light emitting element of the present invention, there are a video camera, a digital camera, a goggle display, a navigation system, an audio reproducing device (e.g., a car audio or audio component set), a personal computer, a game machine, a portable information terminal (e.g., a mobile computer, a mobile phone, a portable game machine, or an electronic book), an image reproducing device provided with a recording medium (specifically, a device for reproducing a recording medium such as a digital versatile disc (DVD) and having a display device for displaying the reproduced image), and the like. Such electronic devices are shown in FIGS. 6A to 6E.

FIG. 6A shows a TV set of the present invention, which includes a housing 9101, a supporting base 9102, a display portion 9103, speaker portions 9104, a video input terminal 9105, and the like. The TV set of the present invention is manufactured by using a light emitting device having a light emitting element of the present invention for the display portion 9103. By using the light emitting device of the present invention, a TV set of the present invention having a long-life display portion can be obtained, and the TV set includes all devices for information display, such as display devices for a computer, television broadcast reception, or advertisement display.

FIG. 6B shows a computer of the present invention, which includes a main body 9201, a housing 9202, a display portion 9203, a keyboard 9204, an external connecting port 9205, a pointing mouse 9206, and the like. The computer of the present invention is manufactured by using a light emitting device having a light emitting element of the present invention for the display portion 9203. By using the light emitting device of the present invention, a computer having a long-life display portion can be obtained.

FIG. 6C shows a goggle display of the present invention, which includes a main body 9301, a display portion 9302, and an arm portion 9303. The goggle display of the present invention is manufactured by using a light emitting device having a light emitting element of the present invention for the display portion 9302. By using the light emitting device of the present invention, a goggle display having a long-life display portion can be obtained.

FIG. 6D shows a mobile phone of the present invention, which includes a main body 9401, a housing 9402, a display portion 9403, an audio input portion 9404, an audio output portion 9405, an operating key 9406, an external connecting port 9407, an antenna 9408, and the like. The mobile phone of the present invention is manufactured by using a light emitting device having a light emitting element of the present invention for the display portion 9403. By using the light emitting device of the present invention, a mobile phone having a long-life display portion can be obtained. Note that by displaying white characters with a black background in the display portion 9403, power consumption of the mobile phone can be suppressed.

FIG. 6E shows a camera of the present invention, which includes a main body 9501, a display portion 9502, a housing 9503, an external connecting port 9504, a remote controller receiving portion 9505, an image receiving portion 9506, a battery 9507, an audio input portion 9508, operating keys 9509, an eye piece 9510, and the like. The camera of the present invention is manufactured by using a light emitting device having a light emitting element of the present invention for the display portion 9502. By using the light emitting device of the present invention, a camera having a long-life display portion can be obtained.

As described above, the light emitting element of the present invention can be applied to a wide range of uses, and this light emitting device can be applied to a various fields of electronic devices, which are electronic devices of the present invention. By using a light emitting device having a light emitting element of the present invention, an electronic device having a long-life display portion can be provided.

### EMBODIMENT 1

An embodiment of a manufacturing method of a phenanthroline derivative compound of the present invention is hereinafter described. However, it is obvious that the present invention is not limited to the embodiment and is specified by description of the scope of claims. In Embodiment 1, three substances of phenanthroline derivative compounds were manufactured, and are represented by General Formulas 2, 3, and 5. Manufacturing Examples 1 to 3 for the three substances are described in order below.

### MANUFACTURING EXAMPLE 1

Manufacturing Example of a Compound Corresponding to General Formula 2

Hereinafter, a manufacturing method of 4,7-diphenyl-2-(2,4,6-trimethylphenyl)-1,10-phenanthroline (hereinafter abbreviated as TMPBP) is described concretely. Under a nitrogen atmosphere, 2,4,6-trimethylphenylmagnesiumbromide (manufactured by Aldrich Chemical Company, 1.0 M, 30 mL) was added into a dried tetrahydrofuran (hereinafter abbreviated as THF) suspension of 4,7-diphenyl-1,10-phenanthroline (manufactured by Tokyo Chemical Industry Co., LTD., 2.0 g, 6.02 mmol) (about 20 mL) to start a reaction, and the reaction mixture was heated under reflux for 24 hours.

Then, the reaction solution was cooled to a room temperature and acidized by being added with hydrochloric acid of 1N. Extraction was performed by using ethyl acetate, an organic layer was dried by using magnesium sulfate and filtered and condensed. A residue was dissolved in chloroform, and this solution was added into silica gel (about 300 mL). Then, the silica gel was cleaned with chloroform, and after cleaning, the silica gel was added with methanol, stirred, and filtered. The obtained methanol solution was condensed, and a residue was dissolved in methylene chloride (about 100 mL). This solution was added with manganese dioxide (manufactured by Sigma Company, 20 g) and stirred for 30 minutes at a room temperature. A reaction mixture was filtered and condensed, and a residue was recrystallized with chloroform-ethyl acetate. Accordingly, TMPBP was obtained with a yield of 76%.

A spectrum data obtained by measuring the obtained compound with NMR is as follows:

¹H NMR (300 MHz, CDCl₃) δ9.25 (d, 1H, J=5Hz), 7.89 (dd, 2H, J=13.6 Hz), 7.45-7.60 (m, 12H), 7.26 (s, 1H), 6.96 (s, 1H), 2.35 (s, 3H), 2.16 (s, 6H).

A structural formula and a synthetic reaction formula of TMPBP fabricated in this manufacturing example are shown in Reaction Formula 2 below.

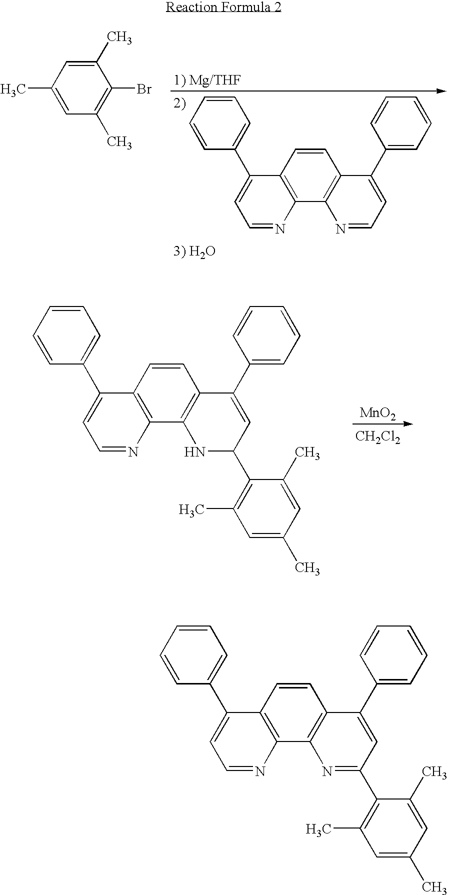

### MANUFACTURING EXAMPLE 2

Manufacturing Example of a Compound Corresponding to General Formula 3

Hereinafter, a manufacturing method of 2-(2-naphthyl)-4,7-diphenyl-1,10-phenanthroline (hereinafter abbreviated as NaBP) is described concretely. Under a nitrogen atmosphere, a dried THF solution of 2-bromonaphthalene (manufactured by Tokyo Chemical Industry Co., LTD., 12.4 g, 60 mmol) was slowly dropped into a mixture of magnesium (manufactured by Kishida Chemical Co., Ltd., 1.46 g, 60 mmol) and dried THF. After dropping, the obtained solution was heated under reflux for two hours, added with a THF solution of 4,7-diphenyl-1,10-phenanthroline, and further heated under reflux for 24 hours. A subsequent purification was conducted in a similar manner as in the case of manufacturing TMPBP of Manufacturing Example 1, and NaBP was obtained with a yield of 29%.

A spectrum data obtained by measuring the obtained compound with NMR is as follows:

¹H NMR (300 MHz, CDCl₃) δ9.31 (d, 1H, J=4Hz), 8.86 (s, 1H), 8.59 (dd, 1H, J=2.9 Hz), 8.22 (s, 1H), 7.45-8.08 (m, 18H): ¹³C NMR (75 MHz, CDCl₃) δ171.1, 156.7, 149.9, 149.3, 148.5, 147.0, 146.9, 138.4, 138.1, 137.0, 133.9, 133.5, 129.7, 129.0, 128.6, 128.5, 128.4, 127.7, 127.5, 126.8, 126.6, 126.2, 125.5, 123.9, 123.8, 123.3, 121.3.

A structural formula and a synthetic reaction formula of NABP fabricated in this manufacturing example are shown in Reaction Formula 3 below.

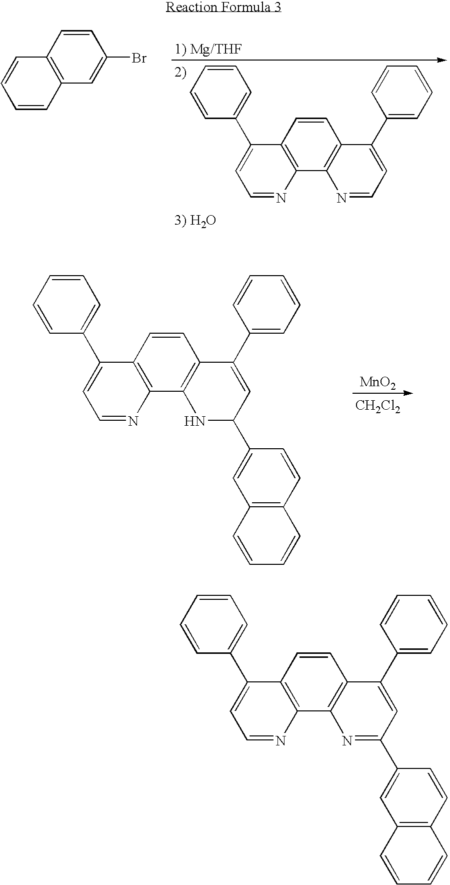

### MANUFACTURING EXAMPLE 3

Manufacturing Example of a Compound Corresponding to General Formula 5.

Hereinafter, a manufacturing method of 2-(9-phenanthryl)-4,7-diphenyl-1,10-phenanthroline (hereinafter abbreviated as PBP) is described concretely. n-butylithium (manufactured by Kanto Chemical., Inc., 1.58N hexane solution, 17 ml, 27 mmol) was dropped in a dried diethyl ether (hereinafter simply abbreviated as ether) suspension of 9-bromophenanthrene (manufactured by Tokyo Chemical Industry Co., LTD., 7.0 g, 27 mmol) (100 mL) at −78° C. After dropping, stirring was performed for one hour at 0° C.

Then, the obtained reaction mixture was added with a dried ether suspension of 4,7-diphenyl-1,10-phenanthroline (100 mL) at 0° C., and after stirring for three hours, water (about 25 mL) was added and extraction was conducted by using methylene chloride. After drying an organic layer by using magnesium sulfate, the organic layer was filtered and condensed, a residue was adsorbed to silica gel, and the silica gel was cleaned with chloroform. After that, the silica gel was extracted by using methanol and acetone, the methanol and the acetone were distilled off, and a residue was recrystallized with chloroform-methanol. Accordingly, PBP of 5.94 g was obtained.

A spectrum data obtained by measuring the obtained compound with NMR is as follows:

¹H NMR (300 MHz, CDCl₃) δ9.25 (d, 2H, J=2.9 Hz), 8.77 (2d, 2H, J=16 Hz), 8.23 (d, 1H, J=8 Hz), 8.18 (s, 1H), 7.45-8.05 (m, 19H): ¹³C NMR (75 MHz, CDCl₃) δ158.9, 149.9, 148.4, 147.0, 146.9, 138.0, 137.7, 131.5, 130.8, 130.5, 129.8, 129.7, 129.4, 129.2, 128.6, 128.5, 128.4, 127.0, 126.74, 126.71, 126.7, 126.4, 125.5, 125.3, 124.0, 123.9, 123.4, 122.9, 122.5.

A structural formula and a synthetic reaction formula of PBP fabricated in this manufacturing example are shown in Reaction Formula 4 below.

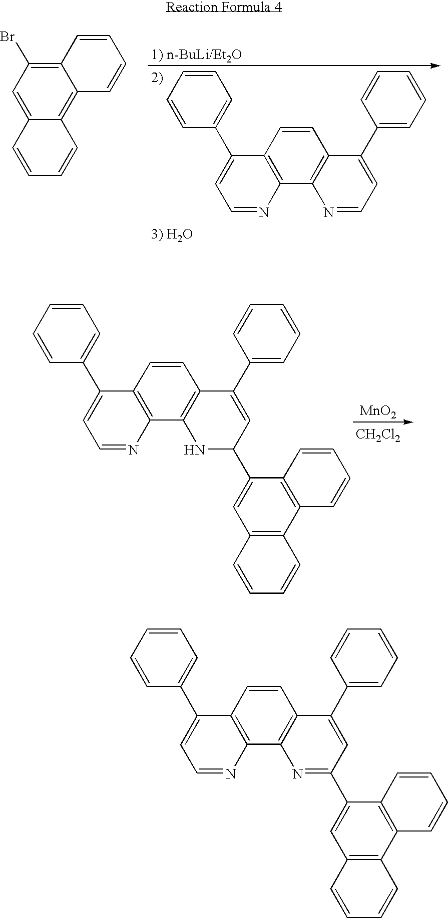

### EMBODIMENT 2

Embodiment 2 describes a result of a performance evaluation test as an electron transporting material performed on the phenanthroline derivative compounds of the present invention, manufactured in Embodiment 1. It is to be noted that the present invention is not limited to the performance evaluation test as an electron transporting material, to be carried out in Embodiment 2, and the results of TMPBP, NaBP, and PBP, which were obtained in Manufacturing Examples 1 to 3 in Embodiment 1. The present invention is specified by the description of the scope of claims.

Oxidation-reduction potentials of the three phenanthroline derivative compounds manufactured in Manufacturing Examples 1 to 3 were measured by using cyclic voltammetry (hereinafter abbreviated as CV). The measurement condition was such that N,N-dimethylformamide was used as a solvent; tetrabutylammonium perchlorate was used as a supporting electrolyte; and a scan speed was 0.1 V/s. Note that Ag was used as a reference electrode at that time. Measurement results thereof are shown in Table 1.

TABLE 1
reduction potentials of phenanthroline derivative compounds

| phenanthroline | |
|---|---|
| derivative | reduction potential |
| compound | (V vs. Ag/AgCl) |
| TMPBP | −2.30 |
| NaBP | −2.22 |
| PBP | −2.27 |

According to the results in Table 1, a peak based on one-electron reduction was observed in each of the three compounds manufactured in the three manufacturing examples. It was also found that, even when a reduction-oxidation cycle of the CV measurement was repeated 200 times, this reduction peak intensity hardly decreased. From this point, it can be said that a compound of the present invention has reversibility to a reduction reaction.

Further, a HOMO level and a LUMO level in film states of the compounds manufactured in the above-described manufacturing examples were measured with the use of a photoelectron spectrometer (manufactured by Riken Keiki Co., Ltd., AC-2) and an ultraviolet-visible light spectrophotometer (manufactured by Japan Spectroscopy Corporation, V550). The results are shown in Table 2. Note that a data obtained by measuring BCP (bathocuproin) is also shown in Table 2, for comparison.

TABLE 2
HOMO level and LUMO level of phenanthroline derivative compounds

| phenanthroline | | | |
|---|---|---|---|
| derivative | HOMO level | LUMO level | band gap |
| compound | (eV) | (eV) | (eV) |
| BCP | −6.10 | −2.53 | 3.57 |
| NaBP | −5.93 | −2.71 | 3.22 |
| PBP | −5.60 | −2.40 | 3.20 |

According to the results in Table 2, a HOMO level of PBP manufactured in Manufacturing Example 3 is positively larger than that of NaBP manufactured in Manufacturing Example 2, and it is speculated that this depends on the difference of a hole injecting property between a naphthalene skeleton and a phenanthrene skeleton. The HOMO level of PBP is positively and considerably larger than that of BCP, and from this point, it can be said that hole injection to PBP is easier than that of BCP. Band gaps of NaBP and PBP are smaller than that of BCP, but those are enough amounts as hosts of light emitting layers for blue color.

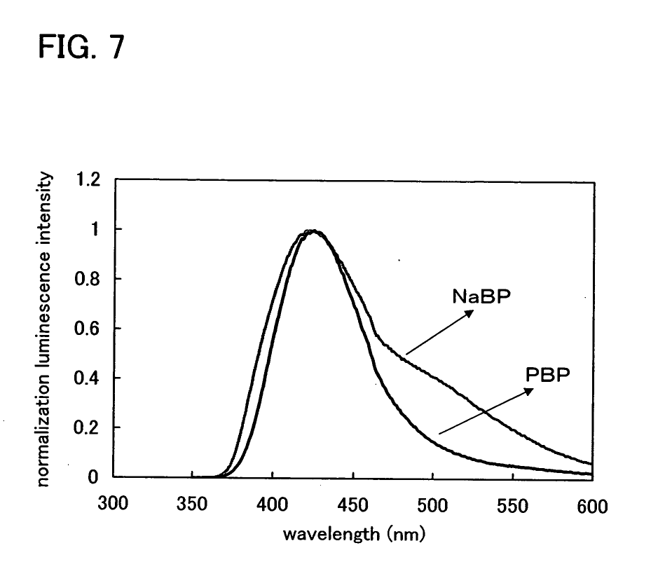

It was confirmed that crystallization was not caused in single films of these NaBP and PBP for a long observation period and that crystallinity of NaBP and PBP could be dramatically suppressed compared with the case of BCP. Further, emission spectrums obtained by measuring NaBP and PBP in film states are shown in FIG. 7. From this observation, it was found that NaBP and PBP had emission maxima of 422 nm and 425 nm respectively and emitted dark blue light.

### EMBODIMENT 3

In Embodiment 3, characteristics of a light emitting element which is manufactured by using PBP manufactured in Manufacturing Example 3 of Embodiment 1 were evaluated. For comparison, a light emitting element using BCP instead of using PBP was manufactured, and a similar evaluation was performed thereto. A film of copper phthalocyanine which was a hole injecting material, and a film of N,N′-di(2-spirofluorenyl)-N,N′-diphenylbenzidine (BSPB) which was a hole transporting material were sequentially formed by a vacuum evaporation method over a film of ITO formed over a glass substrate. The thicknesses thereof are 20 nm and 40 nm respectively.

Next, over these stacked films, a film of PBP, a film of 4,4′-di(N-carbazolyl)-biphenyl (CBP), and a film of 2-t-butyl-9,10-di-(2-naphthyl)anthracene (t-BuDNA) were co-evaporated by evaporation with a thickness of 30 nm. Note that in this co-evaporation, a weight ratio of PBP, CPB, and t-BuDNA was 1:1:1. Over this co-vapor deposition film, a film of Alq which was an electron transporting material was stacked with a thickness of 30 nm, and a film of CaF₂ which was an electron injecting material was formed thereover, and an Al electrode was further formed, so that a light emitting element was manufactured.

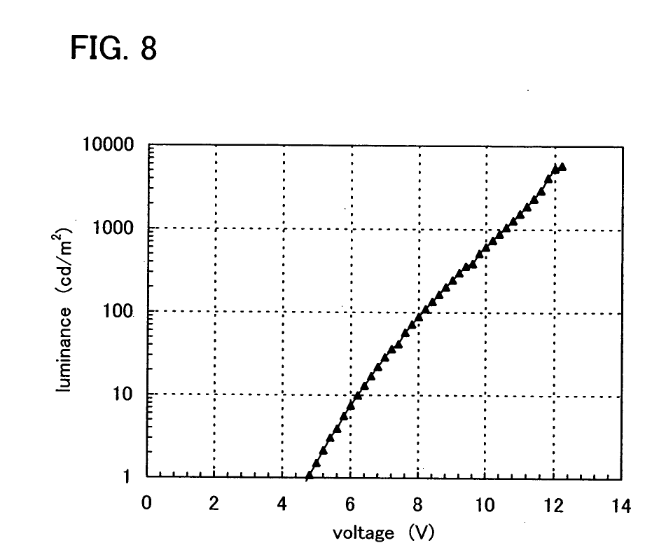

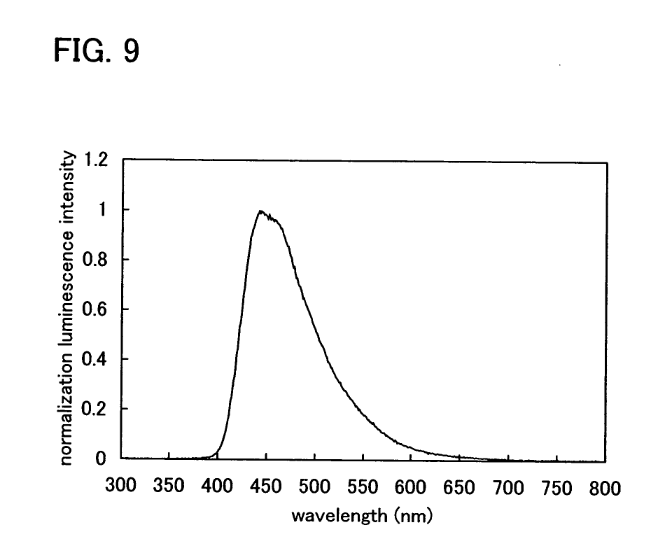

In order to evaluate characteristics of this element, a relation between a voltage and a luminance was measured. The result thereof is shown by a voltage-luminance curve in FIG. 8. According to the result, it was found that light emission started at approximately 4.8 V and the luminance was 600 cd/m² when applying 10 V Further, an emission spectrum was measured. FIG. 9 shows a result thereof. From the result, it can be confirmed that the emission maximum was 450 nm and the light emission with blue color was obtained. In addition, the current efficiency was 1.48 cd/Å at 1000 cd/m², and CIE chromaticity coordinate was measured to be x=0.16 and y=0.14 by using a color luminance meter (manufactured by Topcon Corporation, BM-5A), accordingly, it was found that excellent blue emission could be obtained.

Light Emitting Element Using BCP as Comparative Example and Characteristic Evaluation Thereof

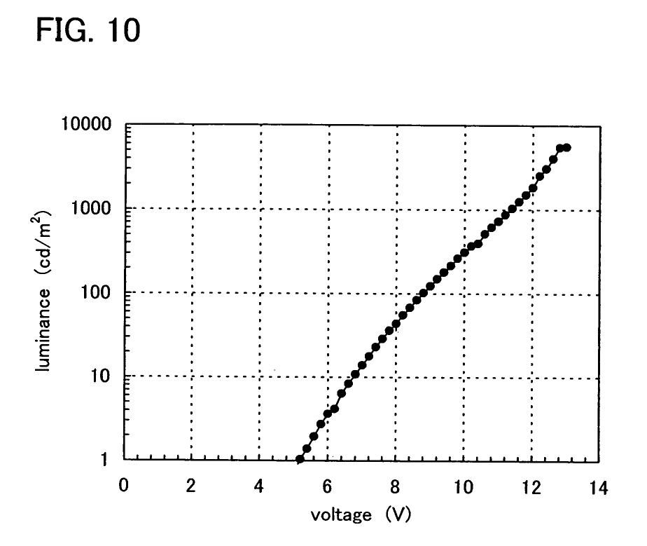

A light emitting element having the same structure except a point that BCP was used instead of PBP, was manufactured, and a performance evaluation test in a similar manner to the light emitting element manufactured using PBP was carried out. A measurement result of a relation between a voltage and a luminance of this element is shown by a voltage-luminance curve in FIG. 10. According to the result, it was found that light emission started at approximately 5.2 V and the luminance was 310 cd/m² when applying 10 V.

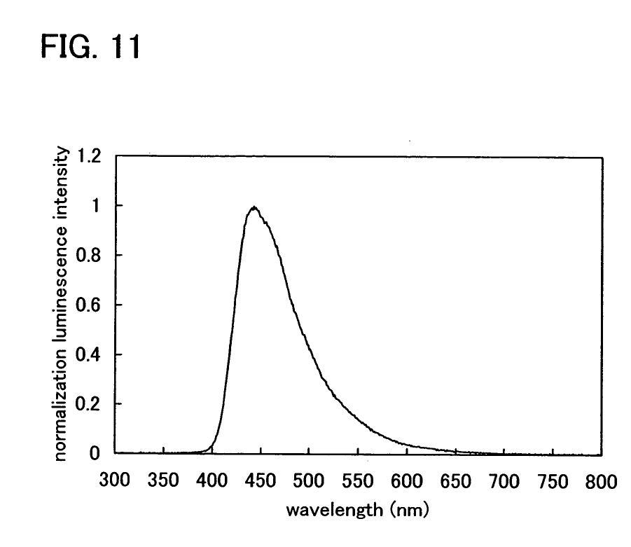

Further, FIG. 11 shows a measurement result of an emission spectrum, and from the result, it can be confirmed that an emission maximum was 450 nm and the light emission with blue color was obtained. In addition, the current efficiency was 1.45 cd/Å at 1000 cd/m², and CIE chromaticity coordinate was measured to be x=0.16 and y=0.16 by using a color luminance meter (manufactured by Topcon Corporation, BM-5A), accordingly, it was found that excellent blue emission can be obtained. From these results, it can be said that PBP exhibits an EL characteristic not so different from that of BCP. In other words, PBP has a similar EL characteristic to BCP, and is yet an excellent material for a light emitting element, which uses a light-emitting organic compound and can overcome the disadvantage of high crystallinity that BCP has.

This application is based on Japanese Patent Application serial no. 2005-233382 filed in Japan Patent Office on Aug. 11, 2005, the entire contents of which are hereby incorporated by reference.

What is claimed is:

1. A phenanthroline derivative compound represented by General Formula 1, wherein Ar¹ represents an aryl group.

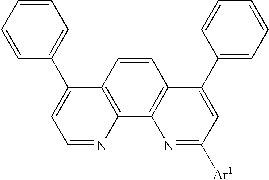

2. A phenanthroline derivative compound represented by General Formula 1, wherein Ar¹ represents a substituted or unsubstituted phenyl group, a substituted or unsubstituted naphthyl group, a substituted or unsubstituted anthryl group, or a substituted or unsubstituted phenanthryl group.

3. A phenanthroline derivative compound represented by General Formula 2, wherein R¹¹ to R¹⁵ each represent hydrogen, an alkyl group, or an aryl group.

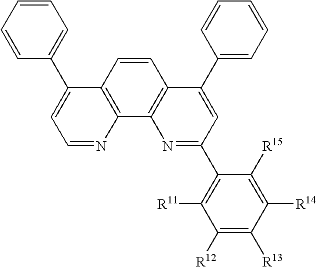

4. A phenanthroline derivative compound represented by General Formula 3 or 4, wherein R²¹ represents hydrogen, an alkyl group, or an aryl group, and R⁴¹ and R⁴² each represent hydrogen, an alkyl group, or an aryl group.

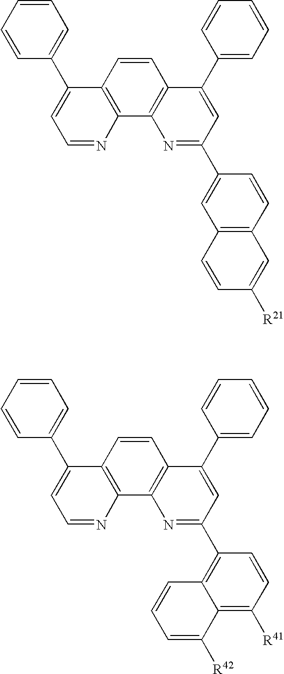

5. A phenanthroline derivative compound represented by General Formula 5 or 6, wherein R³¹, R³², R⁵¹, and R⁵² each represent hydrogen, an alkyl group, or an aryl group.

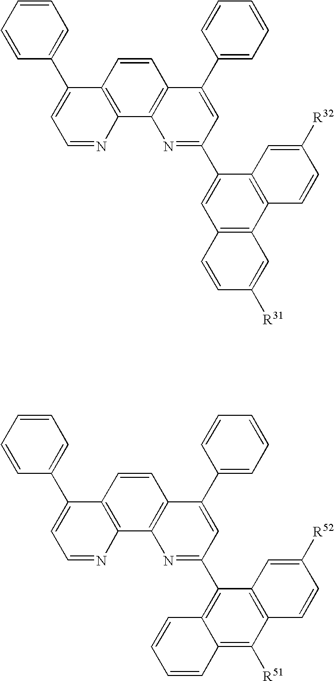

6. A manufacturing method of a phenanthroline derivative compound, comprising the steps of:
reacting aryl halide capable of forming a predetermined aryl group with Mg or BuLi to form a first compound;
reacting the first compound with 4,7-diphenyl-1,10-phenanthroline to form a second compound;
reacting the second compound with water or alcohol to form a third compound; and
oxidizing the third compound by MnO₂ to eliminate hydrogen bonded with nitrogen at a first position of the third compound and hydrogen bonded with carbon at a second position of the third compound.

7. An electron transporting material containing the phenanthroline derivative compound according to claim 1.

8. An electron transporting material containing the phenanthroline derivative compound according to claim 2.

9. An electron transporting material containing the phenanthroline derivative compound according to claim 3.

10. An electron transporting material containing the phenanthroline derivative compound according to claim 4.

11. An electron transporting material containing the phenanthroline derivative compound according to claim 5.

12. A light emitting element including a layer which contains the phenanthroline derivative compound according to claim 1.

13. A light emitting element including a layer which contains the phenanthroline derivative compound according to claim 2.

14. A light emitting element including a layer which contains the phenanthroline derivative compound according to claim 3.

15. A light emitting element including a layer which contains the phenanthroline derivative compound according to claim 4.

16. A light emitting element including a layer which contains the phenanthroline derivative compound according to claim 5.

17. A light emitting element including a light emitting layer which contains the phenanthroline derivative compound according to claim 1.

18. A light emitting element including a light emitting layer which contains the phenanthroline derivative compound according to claim 2.

19. A light emitting element including a light emitting layer which contains the phenanthroline derivative compound according to claim 3.

20. A light emitting element including a light emitting layer which contains the phenanthroline derivative compound according to claim 4.

21. A light emitting element including a light emitting layer which contains the phenanthroline derivative compound according to claim 5.

22. A light emitting element including a light emitting layer which contains a light emitting substance and the phenanthroline derivative compound according to claim 1.

23. A light emitting element including a light emitting layer which contains a light emitting substance and the phenanthroline derivative compound according to claim 2.

24. A light emitting element including a light emitting layer which contains a light emitting substance and the phenanthroline derivative compound according to claim 3.

25. A light emitting element including a light emitting layer which contains a light emitting substance and the phenanthroline derivative compound according to claim 4.

26. A light emitting element including a light emitting layer which contains a light emitting substance and the phenanthroline derivative compound according to claim 5.

27. A light emitting element including an electron transporting layer which contains the phenanthroline derivative compound according to claim 1.

28. A light emitting element including an electron transporting layer which contains the phenanthroline derivative compound according to claim 2.

29. A light emitting element including an electron transporting layer which contains the phenanthroline derivative compound according to claim 3.

30. A light emitting element including an electron transporting layer which contains the phenanthroline derivative compound according to claim 4.

31. A light emitting element including an electron transporting layer which contains the phenanthroline derivative compound according to claim 5.

32. A light emitting element including a blocking layer which contains the phenanthroline derivative compound according to claim 1.

33. A light emitting element including a blocking layer which contains the phenanthroline derivative compound according to claim 2.

34. A light emitting element including a blocking layer which contains the phenanthroline derivative compound according to claim 3.

35. A light emitting element including a blocking layer which contains the phenanthroline derivative compound according to claim 4.

36. A light emitting element including a blocking layer which contains the phenanthroline derivative compound according to claim 5.

37. A light emitting device including the light emitting element according to claim 12.

38. A light emitting device including the light emitting element according to claim 13.

39. A light emitting device including the light emitting element according to claim 14.

40. A light emitting device including the light emitting element according to claim 15.

41. A light emitting device including the light emitting element according to claim 16.

42. A light emitting device including the light emitting element according to claim 17.

43. A light emitting device including the light emitting element according to claim 18.

44. A light emitting device including the light emitting element according to claim 19.

45. A light emitting device including the light emitting element according to claim 20.

46. A light emitting device including the light emitting element according to claim 21.

47. A light emitting device including the light emitting element according to claim 22.

48. A light emitting device including the light emitting element according to claim 23.

49. A light emitting device including the light emitting element according to claim 24.

50. A light emitting device including the light emitting element according to claim 25.

51. A light emitting device including the light emitting element according to claim 26.

52. A light emitting device including the light emitting element according to claim 27.

53. A light emitting device including the light emitting element according to claim 28.

54. A light emitting device including the light emitting element according to claim 29.

55. A light emitting device including the light emitting element according to claim 30.

56. A light emitting device including the light emitting element according to claim 31.

57. A light emitting device including the light emitting element according to claim 32.

58. A light emitting device including the light emitting element according to claim 33.

59. A light emitting device including the light emitting element according to claim 34.

60. A light emitting device including the light emitting element according to claim 35.

61. A light emitting device including the light emitting element according to claim 36.

62. An electronic device including the light emitting element according to claim 12.

63. An electronic device including the light emitting element according to claim 13.

64. An electronic device including the light emitting element according to claim 14.

65. An electronic device including the light emitting element according to claim 15.

66. An electronic device including the light emitting element according to claim 16.

67. An electronic device including the light emitting element according to claim 17.

68. An electronic device including the light emitting element according to claim 18.

69. An electronic device including the light emitting element according to claim 19.

70. An electronic device including the light emitting element according to claim 20.

71. An electronic device including the light emitting element according to claim 21.

72. An electronic device including the light emitting element according to claim 22.

73. An electronic device including the light emitting element according to claim 23.

74. An electronic device including the light emitting element according to claim 24.

75. An electronic device including the light emitting element according to claim 25.

76. An electronic device including the light emitting element according to claim 26.

77. An electronic device including the light emitting element according to claim 27.

78. An electronic device including the light emitting element according to claim 28.

79. An electronic device including the light emitting element according to claim 29.

80. An electronic device including the light emitting element according to claim 30.

81. An electronic device including the light emitting element according to claim 31.

82. An electronic device including the light emitting element according to claim 32.

83. An electronic device including the light emitting element according to claim 33.

84. An electronic device including the light emitting element according to claim 34.

85. An electronic device including the light emitting element according to claim 35.

86. An electronic device including the light emitting element according to claim 36.
